(19) United States
(12) Patent Application Publication
(10) Pub. No.: US 2025/0099655 A1
(43) Pub. Date: Mar. 27, 2025

(54) ARTIFICIAL SKIN COMPRISING FIBROUS MAT

(71) Applicant: NANOSPUN TECHNOLOGIES LTD., Yokneam (IL)

(72) Inventors: Alon ARMELIN, Gilon (IL); Ohad BENDROR, Los Altos, CA (US)

(73) Assignee: NANOSPUN TECHNOLOGIES LTD., Yokneam (IL)

(21) Appl. No.: 18/728,203

(22) PCT Filed: Jan. 11, 2023
(86) PCT No.: PCT/IL2023/050036

§ 371 Date: Jul. 11, 2024

Related U.S. Application Data
(60) Provisional application No. 63/298,549, filed on Jan. 11, 2022.
(60) Provisional application No. 63/434,805, filed on Dec. 22, 2022.

(51) Int. Cl. A61L 27/60; A61L 27/16; A61L 27/20; A61L 27/36; A61L 27/54; A61L 27/56

(52) U.S. Cl. CPC A61L 27/60; A61L 27/16; A61L 27/20; A61L 27/3691; A61L 27/54; A61L 27/56; A61L 2400/18; A61L 2430/34

(57) ABSTRACT

There is provided a fibrous mat comprising a plurality of electrospun fibers, wherein each of the electrospun fibers comprising a shell and a core comprising a water-soluble polymer and a plurality of cells, e.g., dormant cells or active cells. Further provided are methods of activating the dormant cells and method of use thereof, such as for supplementing a skin of the subject with a cosmeceutical ingredient.

### CROSS-REFERENCE TO RELATED APPLICATIONS

This application is a PCT International Application claiming the benefit of priority of U.S. Patent Application No. 63/298,549, filed Jan. 11, 2022, and of U.S. Patent Application No. 63/434,805, filed Dec. 22, 2022, which are hereby incorporated by reference in their entirety.

### FIELD OF THE INVENTION

The present invention, in some embodiments thereof, relates to a fibrous mat encapsulating viable cells, preparation and use thereof.

### BACKGROUND OF THE INVENTION

Electrospun fibrous mats encapsulating cells have been previously disclosed. However, there remains an unmet need of encapsulating very high load of cells within the fibers, while maintaining the integrity of the fiber.

As a non-limiting example, the skin's biologically active ecosystem, the bacteria located at the human skin, is known as the skin microbiome, and its existence and activity are vital to guard and support skin health including boosting skin hydration and elasticity, preventing skin sensitivity and redness, reducing wrinkles, and helping anti-aging effects, to name just a few. However, the human skin microbiome is constantly subjected to environmental damage. Furthermore, exposure of the skin to various skin care products can pose a threat to the skin microbiome, thus affecting skin health. To this end, there is no available solution providing an effective amount or concentration of living/viable microbial cells, sufficient for implementation thereof in skin and/or topical care.

Accordingly, there is an unmet need for a user-friendly skin-care article with a prolonged shelf-life, and comprising an effective amount of viable cells, which can be easily activated in-situ prior to application thereof on the human skin.

### SUMMARY OF THE INVENTION

In one aspect of the invention, there is provided a fibrous mat comprising a plurality of electrospun fibers, wherein each of the electrospun fibers comprises a shell encapsulating a core, wherein the fibrous mat is characterized by a thickness of between 10 and 2000 um; and the core comprises a hydrophilic polymer and a plurality of cells.

In another aspect of the invention, there is provided a fibrous mat comprising a plurality of electrospun fibers, wherein each of the electrospun fibers comprising a shell encapsulating a core, wherein: the fibrous mat is characterized by a thickness of between 10 and 2000 um; the core comprises (i) a water-soluble material comprising a water-soluble polymer, and (ii) a plurality of cells; the shell is a porous shell comprising a water insoluble polymer; and wherein a loading of the plurality of cells within the fibrous mat is up to 10¹³ CFU/cm².

In one embodiment, the plurality of cells is selected from microbial cells, microbial spores, a microorganism, or a combination thereof; optionally wherein the microbial cells or microbial spores comprise one or more species of bacteria, fungi, or both. In one embodiment, the cells are selected from microbial cells, microbial spores, or both. In one embodiment, the cells are dormant cells. In one embodiment, the cells are live cells.

In one embodiment, the fibrous mat is shapeable, configured to substantially obtain a shape of the application site and to retain on the application site for a predetermined time period.

In one embodiment, a dry weight per weight (w/w) concentration of the dormant cells within the mat is up to 95%. In one embodiment, a dry weight per weight (w/w) concentration of the dormant cells within the mat is up to 90%. In one embodiment, a dry weight per weight (w/w) concentration of the dormant cells within the mat is up to 80%.

In one embodiment, an average cross-section of the electrospun fibers is between 1 and 500 um. In one embodiment, an average cross-section of the electrospun fibers is between 1 and 300 um. In one embodiment, an average cross-section of the electrospun fibers is between 1 and 200 um. In one embodiment, an average cross-section of the electrospun fibers is between 1 and 100 um.

In one embodiment, a loading of the plurality of cells within the fibrous mat is between 10⁹ and 10¹² CFU/cm².

In one embodiment, the fibrous mat is composed essentially of biocompatible materials, cosmeceutical grade materials, or medical grade materials, wherein the shell is a solid porous shell, comprising a hydrophobic polymer comprising a fluoropolymer including any copolymer thereof.

In one embodiment, the water insoluble polymer comprises a water insoluble polysaccharide, a fluoropolymer, or PVDF-HFP, including any combination and any copolymer thereof.

In one embodiment, the water insoluble polysaccharide comprises cellulose, a water insoluble cellulose derivative, including any combination and any copolymer thereof. In one embodiment, the water insoluble cellulose derivative comprises cellulose acetate, cellulose acetate phthalate, methyl cellulose, ethyl cellulose, ethyl methyl cellulose, hydroxy ethyl cellulose, hydroxy propyl cellulose, carboxy methyl cellulose, hydroxypropyl methyl cellulose, including any combination and any copolymer thereof.

In one embodiment, the water-soluble polymer is selected from a water-soluble polysaccharide, a polyol, a polyvinyl alcohol, polyalkyleneoxide, PVP, a polyether, including any copolymer and any combination thereof.

In one embodiment, the shell comprises up to 90% by weight of the hydrophobic polymer, wherein the hydrophobic polymer comprises PVDF-HFP, and/or cellulose.

In one embodiment, the shell is substantially or completely composed of natural polymers.

In one embodiment, the fibrous mat is characterized by adhesiveness to a human skin, wherein the fibrous mat is in a moist form.

In one embodiment, the microbial cells comprise one or more species of bacteria, yeast, fungi or any combination thereof. In one embodiment, the microbial cells comprises probiotic bacteria.

In one embodiment, the fibrous mat further comprising a plant extract, a bacterial metabolite, a fungal metabolite, yeast metabolite or any combination thereof.

In one embodiment, the fibrous mat is characterized by a moisture content of less than 10%, and wherein the dormant cells maintain their viability within the mat for at least 7 days, at least 14 days, or at least 30 days.

In one embodiment, the fibrous mat is characterized by water absorption capability of at least 100%, relative to the dry weight of said fibrous mat.

In one embodiment, the fibrous mat is in a form of a non-woven uniform layer.

In one embodiment, the fibrous mat is in a form of topical product, such as configured for application to a target site on a skin of a subject.

In one embodiment, the fibrous mat is for use in any one of cosmeceutical, beauty, hygiene, and skincare use.

In one embodiment, the topical product is further configured for subsequent removal thereof from the target site, and wherein the topical product has sufficient mechanical strength to remain intact upon removal thereof.

In one embodiment, the topical product is configured to substantially adopt a shape of the target site.

In one embodiment, a length dimension, a width dimension or both of the topical product is substantially compatible with the dimension of the target site or any part thereof.

In one embodiment, the topical product is configured for stably adhere to the target site for a predetermined time period.

In another aspect, there is provided a kit comprising the fibrous mat of the invention packaged within a container, wherein the fibrous mat is characterized by a water content of at most 20%, or at most at most 10%, and wherein a wall of the container is water impermeable and is substantially oxygen impermeable.

In one embodiment, the wall of the container is stable at a temperature up to 60° C. and is characterized by a sufficient heat transfer capacity.

In one embodiment, the kit further comprises a water-tight container comprising an activating composition, wherein the activating composition provides conditions for returning the dormant cells to an active state (e.g., germination).

In one embodiment, the kit further comprises a water-tight container comprising an activating composition, wherein the activating composition is capable of boosting the live cells to secrete elements and/or metabolites such as vitamins, acids, and proteins.

In one embodiment, the activating composition is a liquid comprising an effective amount of a plant-based compound, or a plant extract, and optionally any one of a nutrient, a mineral, a sugar (mono-, di-, oligo-, and/or polysaccharide), a surface active compound or any combination thereof. In one embodiment the activating composition is a natural composition.

In one embodiment the activating composition is preservative free.

In one embodiment, the activating composition is characterized by pH between 3 and 10.

In one embodiment, the effective amount is sufficient for activation of dormant cells upon contacting the activating composition with the fibrous mat under operable conditions.

In one embodiment, the operable conditions comprise a temperature between about 15 and about 60° C. In one embodiment, the operable conditions comprise a temperature between about 30 and about 60° C.

In one embodiment, the wall of the container defines a lumen configured to hold a liquid volume.

In one embodiment, the container further comprises seal, and wherein the seal is a removable seal or a breakable seal.

In one embodiment, the seal is characterized by an open state and by a closed state.

In one embodiment, the seal is in a form of a valve, and wherein in the open state the valve is configured to be in fluid communication with the water-tight container, and to support a liquid flow from the water-tight container into the lumen.

In one embodiment, the valve in the closed state is configured to seal the container.

In one embodiment, the fibrous mat is placed within an internal container residing within an external container comprising the activating composition.

In one embodiment, the kit includes one or more additional compartments such as for containing a designated pre and/or post treatment solutions.

In one embodiment, the kit further comprises a heating device.

In another aspect, there is provided a method for in-situ generation of a cell metabolite, the method comprising contacting the fibrous mat of the invention with an activating composition under appropriate conditions sufficient for activating the dormant cells and/or improving their activity, thereby inducing in-situ generation of the cell metabolite; wherein the activating composition is a liquid comprising capable of providing the dormant cells from the dormant state into an active state.

In another aspect, there is provided a method comprising providing the kit the kit of the invention and contacting the fibrous material of the kit with an activating composition of the invention under appropriate conditions, thereby obtaining a topical product comprising a metabolite; and applying the topical product at a target site on the skin of a subject, thereby supplementing the skin with the metabolite.

In one embodiment, the topical product comprises the mat in a moist state. In one embodiment, the topical product is a solution extracted and/or released from the mat.

In one embodiment, the activating composition comprises an effective amount of a plant-based compound, or a plant extract, and optionally any one of a nutrient, a mineral, a sugar (mono-, di-, oligo-, and/or polysaccharide), a surface-active compound or any combination thereof.

In one embodiment, the activating composition comprises an effective amount of a non-plant-based compound.

In one embodiment, the activating composition is characterized by pH between 3 and 10.

In one embodiment, contacting comprises soaking of the fibrous mat within the activating composition.

In one embodiment, appropriate conditions comprise a temperature between about 15 and about 60° C. In one embodiment, appropriate conditions comprise a temperature between about 30 and about 55° C.

In one embodiment, the topical product is provided as a wet and/or moisturized mat.

In one embodiment the mat is degradable, such as by applying to a subject's skin.

In one embodiment the mat may be comprised from more than one kind of fibers (i.e., different type of fibers wherein each type of fiber has a specific property such as a biological property, chemical property and/or physical property).

In some embodiment the met may include, such as prior to the activation step, active biological molecules including but not limited to enzymes, hormones and growth factors.

In another aspect, there is provided a fibrous mat comprising a plurality of electrospun fibers, wherein each of the electrospun fibers comprising a shell encapsulating a core, wherein the fibrous mat is characterized by a thickness of between 10 and 2000 um; the core comprises (i) a water-soluble material comprising a water-soluble polymer, and (ii) a plurality of cells; and the shell is a porous shell comprising a water insoluble polymer; and wherein a loading of the plurality of cells within the fibrous mat is up to 10¹³.

In one embodiment, the water-soluble polymer is selected from a water-soluble polysaccharide, a polyol, a polyvinyl alcohol, polyalkyleneoxide, PVP, a polyether, including any copolymer and any combination thereof.

In one embodiment, the water insoluble cellulose derivative comprises cellulose acetate, cellulose acetate phthalate, methyl cellulose, ethyl cellulose, ethyl methyl cellulose, hydroxy ethyl cellulose, hydroxy propyl cellulose, carboxy methyl cellulose, hydroxypropyl methyl cellulose, including any combination and any copolymer thereof.

In one embodiment, the water-soluble material further comprises a monosaccharide, a di-saccharide, an oligosaccharide, or any combination thereof.

In one embodiment, the fibrous mat is characterized by water absorption capability of up to 1000%, relative to the dry weight of said fibrous mat.

In one embodiment, an average cross-section of the electrospun fibers within the fibrous mat is between about 10 and about 200 um; wherein the w/w concentration of the plurality of cells within the mat is up to 90%, or wherein a loading of the plurality of cells within the fibrous mat is up to 10¹² CFU/cm²; wherein the shell comprises cellulose, or the water insoluble cellulose derivative; and wherein the core comprises (i) at least one of a monosaccharide, a di-saccharide, and an oligosaccharide; and (ii) the water-soluble polymer selected from polyalkyleneoxide and the water soluble polysaccharide.

In another aspect, there is provided a fibrous substrate comprising polymeric fibers, each polymeric fiber is an electrospun fiber comprising a shell encapsulating a core, wherein the core comprises and a plurality of cells and a water-soluble material; the shell comprises a water-insoluble polymer; an average cross-section of the polymeric fiber is between 1 and 500 um; and wherein a loading of said plurality of cells within the fibrous substrate is between 10⁵ and 10¹³ CFU/cm².

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1 is an image presenting a schematic illustration of an exemplary kit comprising the dry fibrous mat of the invention within the air-tight package in contact with an exemplary heating device.

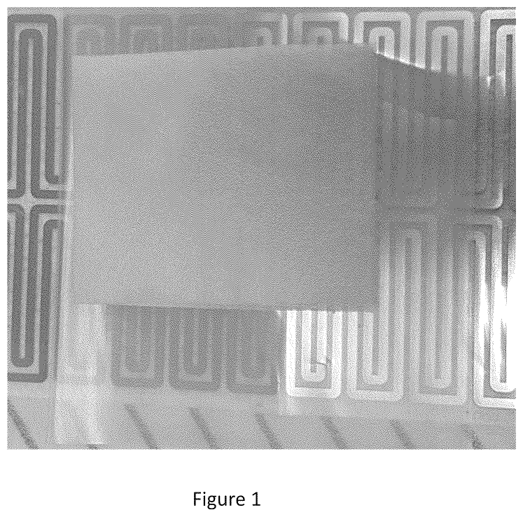

FIG. 2 is an image presenting an exemplary fibrous mat of the invention.

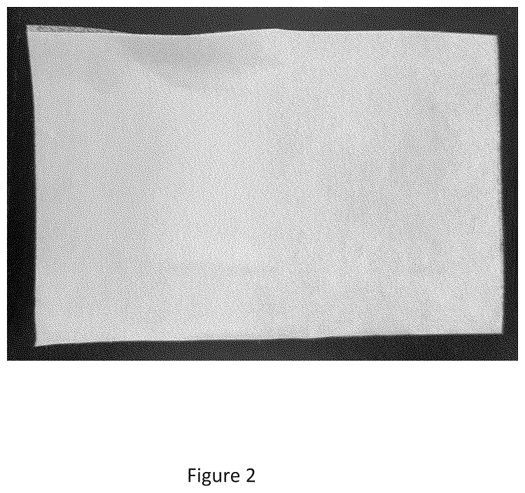

FIG. 3 is a SEM micrograph of an exemplary fibrous mat of the invention.

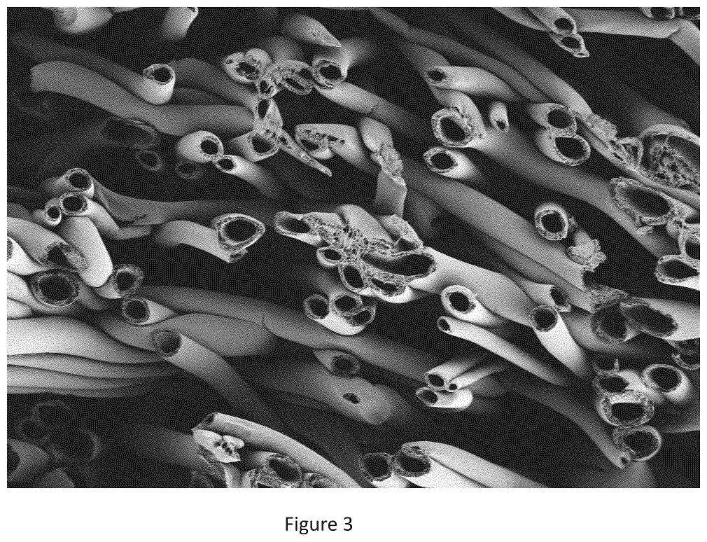

### DETAILED DESCRIPTION OF THE INVENTION

In one aspect, there is provided a fibrous material (e.g. mat) in a form of a fibrous matrix, wherein the fibrous matrix comprises polymeric fibers, wherein the polymeric fibers are electrospun fibers comprising a hydrophobic (or water insoluble) porous shell and a hydrophilic (or water soluble) core.

In another aspect, there is provided a fibrous material in a form of a fibrous matrix (e.g., a mat), wherein the fibrous matrix comprises polymeric fibers and plurality of cells encapsulated therewithin. In some embodiments, the fibrous material is in a form of one or more layers comprising a plurality of micron-sized polymeric fibers, wherein each polymeric fiber comprises a polymeric shell encapsulating a core, wherein the core comprises a plurality of live and/or dormant cells, and/wherein the polymeric shell is a single layer shell. In some embodiments, the polymeric shell is in direct contact (or is directly bound) to the core. In some embodiments, the polymeric shell is devoid of an inner coating layer at the interphase between the core and the shell. In some embodiments, the fibrous material comprises or is essentially composed of electrospun fibers. In some embodiments, a w/w content of the electrospun fibers in the fibrous material of the invention is between 80 and 100%, between 85 and 99%, between 90 and 99%, between about 90 and 100%, including any range between. In some embodiments, the fibrous material is in a solid state at a temperature up to 100° C., up to 200° C., or more.

As exemplified herein, the invention in some embodiments thereof, is based on the surprising finding that a particular core composition of the electrospun fibers is capable of supporting high loading of viable cells, so as to result in a fibrous mat encapsulating about 50% and up to about 95% of viable cells (by dry weight of the fibrous mat).

The invention, in some embodiments thereof, is also based on the surprising finding that particular core compositions of the electrospun fibers of the invention are capable of supporting a shelf-life of at least one month under ambient conditions (e.g. between 1° and 30° C.) or even longer shelf life at cold storage conditions (e.g. between −20 and 5° C.). In particular, the inventors observed that by implementing a core electrospinning solution containing at least 0.5 g/ml of a water-soluble polymeric thickener (e.g. a mono-, and/or di-saccharides, a water-soluble polysaccharide, or a water-soluble polymer) resulted in electrospun microfibers with an exceptionally high loading of the encapsulated cells. Furthermore, the core composition disclosed herein, have been found beneficial for retaining viability of the encapsulated cells for a time period of at least about 1-3 months, or more, when stored in an airtight container. The encapsulated cells have been subsequently activated by integrating and/or soaking the dry mat with an activation solution, under suitable conditions. The cell activity has been assessed by monitoring the synthesis rate or concentration of the cell metabolites.

According to one aspect, the fibrous mat comprises a plurality of electrospun fibers, wherein each of the electrospun fibers comprising a shell encapsulating a core, wherein: the fibrous mat is characterized by a thickness of between 10 and 2000 um; the core comprises (i) a water-soluble material comprising a water-soluble polymer, and (ii) a plurality of cells; the shell is a porous shell comprising a water insoluble polymer; and wherein a loading of the plurality of cells within the fibrous mat is up to 10¹³ CFU/cm².

According to some embodiments, the plurality of cells is selected from microbial cells, microbial spores, a microorganism, or a combination thereof; optionally wherein the microbial cells or microbial spores comprise one or more species of bacteria, fungi, or both.

According to some embodiments, a dry weight per weight (w/w) concentration of the plurality of cells within the mat is up to about 95%.

According to some embodiments, the w/w concentration of the plurality of cells within the mat is up to 90%, optionally wherein the plurality of cells are dormant cells.

According to some embodiments, an average cross-section of the electrospun fibers within the fibrous mat is between 1 and 500 um. According to some embodiments, an average cross-section of the electrospun fibers within the fibrous mat is between 10 and 300 um.

According to some embodiments, the loading of the plurality of cells within the fibrous mat is between 10⁹ and 10¹² CFU/cm².

According to some embodiments, the fibrous mat is composed essentially of biocompatible materials or cosmeceutical grade materials.

According to some embodiments, said shell comprises at least 50% of the water insoluble polymer by dry weight, and optionally further comprises a water-soluble polymer.

According to some embodiments, the water insoluble polymer comprises a water insoluble polysaccharide, a fluoropolymer, or PVDF-HFP, including any combination and any copolymer thereof.

According to some embodiments, the water insoluble polysaccharide comprises cellulose, a water insoluble cellulose derivative, including any combination and any copolymer thereof.

According to some embodiments, the water insoluble cellulose derivative comprises cellulose acetate, cellulose acetate phthalate, methyl cellulose, ethyl cellulose, ethyl methyl cellulose, hydroxy ethyl cellulose, hydroxy propyl cellulose, carboxy methyl cellulose, hydroxypropyl methyl cellulose, including any combination and any copolymer thereof.

According to some embodiments, the water-soluble polymer is selected from a water-soluble polysaccharide, a polyol, a polyvinyl alcohol, polyalkyleneoxide, PVP, a polyether, including any copolymer and any combination thereof.

According to some embodiments, the water-soluble material further comprises a monosaccharide, a di-saccharide, an oligosaccharide, or any combination thereof.

According to some embodiments, the fibrous mat is shapeable, and is characterized by water absorption capability of up to 1000%, relative to the dry weight of said fibrous mat.

According to some embodiments, the fibrous mat is in a moist form and is characterized by adhesiveness to a human skin.

According to some embodiments, the fibrous mat further comprises a plant extract, a bacterial metabolite, a fungal metabolite, or any combination thereof.

According to some embodiments, the mat is characterized by a moisture content of less than 10%, and wherein the dormant cells maintain their viability within the mat for at least 3 months.

According to some embodiments, an average cross-section of the electrospun fibers within the fibrous mat is between about 10 and about 200 um; wherein the w/w concentration of the plurality of cells within the mat is up to 90%, or wherein a loading of the plurality of cells within the fibrous mat is up to 10¹² CFU/cm²; wherein the shell comprises cellulose, or the water insoluble cellulose derivative; and wherein the core comprises (i) at least one of a monosaccharide, a di-saccharide, and an oligosaccharide; and (ii) the water-soluble polymer selected from polyalkyleneoxide and the water soluble polysaccharide.

According to some embodiments, the water-soluble polysaccharide is a water-soluble gum, and wherein the polyalkyleneoxide is PEG.

According to some embodiments, the fibrous mat is in a form of a non-woven uniform layer. According to some embodiments, the mat is in a form of a sphere. According to some embodiments, the mat is in a form of a topical product configured for application to a target site on a skin of a subject.

According to some embodiments, the topical product is further configured for subsequent removal thereof from the target site, and wherein the topical product has sufficient mechanical strength to remain intact upon removal thereof.

According to some embodiments, the topical product is characterized by a tensile strength of at least 1 MPa.

According to some embodiments, the topical product is configured to substantially adopt a shape of the target site.

According to some embodiments, a length dimension, a width dimension or both of the topical product is substantially compatible with the dimension of the target site.

According to some embodiments, the topical product is configured for stably adhere to the target site for a predetermined time period.

According to another aspect, there is provided a kit comprising the fibrous mat of the invention packaged within a container, wherein the fibrous mat is characterized by a water content of at most 10%, and wherein a wall of the container is water impermeable and is substantially oxygen impermeable.

According to some embodiments, the wall of the container is stable at a temperature up to 60° C. and is characterized by a sufficient heat transfer capacity.

According to some embodiments, the kit further comprises a water-tight container comprising an activating composition, wherein the plurality of cells within the fibrous mat are substantially in a dormant state, and wherein the activating composition is capable of providing the plurality of cells from a dormant state into an active state.

According to some embodiments, the activating composition is a liquid comprising an effective amount of a plant-based compound, or a plant extract, and optionally any one of a nutrient, a mineral, a mono-saccharide, a disaccharide, oligo-saccharide, a polysaccharide, a surface-active compound, or any combination thereof.

According to some embodiments, the activating composition is characterized by pH between 3 and 10.

According to some embodiments, the effective amount is sufficient for activation of dormant cells upon contacting the activating composition with the fibrous mat under operable conditions.

According to some embodiments, the operable conditions comprise a temperature between about 30 and about 55° C.

According to some embodiments, the wall of the container defines a lumen configured to hold a liquid volume; and wherein the container further comprises seal, and wherein the seal is a removable seal or a breakable seal.

According to some embodiments, the seal is characterized by an open state and by a closed state; wherein the seal is in a form of a valve, and wherein in the open state the valve is configured to be in fluid communication with the water-tight container, and to support a liquid flow from the water-tight container into the lumen wherein in the closed state the valve is configured to seal the container.

According to some embodiments, the kit further comprising a heating device.

According to some embodiments, the kit further comprises instructions for contacting the fibrous mat with the activating composition, to obtain a moist mat, and applying the moist mat to a skin of a subject.

According to some embodiments, the kit further comprising any one of: (i) instructions for contacting the fibrous mat with the activating composition, to obtain a moist mat, and applying extracts from the moist mat to a skin of a subject; and (ii) instructions further comprise heating the moist mat, and wherein said heating is performed prior to skin application or subsequent to skin application.

According to some embodiments, the heating is by the heating device.

According to another aspect there is provided a method for in-situ generation of a cell metabolite, the method comprising contacting the fibrous mat of the invention with an activating composition under appropriate conditions sufficient for activating the dormant cells, thereby inducing in-situ generation of the cell metabolite; wherein the activating composition is a liquid comprising capable of providing the dormant cells from the dormant state into an active state.

According to another aspect there is provided a method comprising: providing the kit of the invention and contacting the fibrous mat with an activating composition under appropriate conditions, thereby obtaining a topical product comprising a metabolite; and applying the product at a target site on the skin of a subject, thereby supplementing the skin with the metabolite.

According to some embodiments, the topical product comprises the mat in a moist state.

According to some embodiments, the activating composition comprises an effective amount of a plant-based compound, or a plant extract, and optionally any one of a nutrient, a mineral, a sugar, a surface-active compound or any combination thereof.

According to some embodiments, the activating composition is characterized by pH between 3 and 10.

According to some embodiments, contacting comprises soaking of the fibrous mat within the activating composition.

According to some embodiments, appropriate conditions comprise a temperature between about 30 and about 55° C.

According to some embodiments, appropriate conditions comprise contacting the moist mat with (i) the heating device, (ii) with the skin of the subject or both (i) and (ii).

According to some embodiments, said applying is under conditions sufficient for providing the moist mat to a temperature between about 30 and about 55° C.

According to some embodiments, said providing is performed in-situ on the skin of the subject.

According to some embodiments, said providing is by contacting the moist mat with a heating device, and wherein said contacting is performed in-situ on the skin of the subject.

According to another aspect there is provided a fibrous substrate comprising polymeric fibers, each polymeric fiber is an electrospun fiber comprising a shell encapsulating a core, wherein: the core comprises and a plurality of cells and a water-soluble material; the shell comprises a water-insoluble polymer; an average cross-section of the polymeric fiber is between 1 and 500 um; and wherein a loading of said plurality of cells within said fibrous substrate is at least 10E5 units per cm².

According to some embodiments, the water-soluble material comprises a water-soluble polymer selected from a polysaccharide, a polyol, a polyvinyl alcohol, a polyether and any combination thereof.

According to some embodiments, the water-soluble material further comprises a monosaccharide, a di-saccharide, an oligosaccharide, or any combination thereof.

According to some embodiments, a w/w ratio between the shell and core within the fiber is between 50:1 and 1:50, and wherein a dry w/w concentration of the plurality of cells within the fibrous substrate is between 40 and 98%.

According to some embodiments, said fibrous substrate is in a form of a fibrous mat.

According to some embodiments, said fibrous mat is an electrospun mat characterized by a thickness of between 10 and 2000 um.

According to some embodiments, the water-insoluble polymer is selected from cellulose, a fluoropolymer, PVDF-HFP, PVP, PCL, PLA, PGA, polystyrene, PES, an acrylate polymer, a water insoluble polysaccharide, a water insoluble cellulose derivative, including any combination and any co-polymer thereof.

According to some embodiments, the water-insoluble polymer constitutes at least 50% by dry weight of said shell; and wherein the shell is characterized by a plurality of pores characterized by an average pore size between 10 and 500 nm.

According to some embodiments, the shell is characterized by a thickness of between 10 nm and about 10 um.

According to some embodiments, the water-insoluble polymer is characterized by solubility of at least 5% w/w in a solvent selected from THF, DMF, acetone, DMAC, chloroform, DMSO, DCM, NMP, TCE, TFE, butanol, methanol, ethanol, HFP, Isopropanol, Ethyl acetate, Ethanolamine, pentane, ethylene glycol, Diethyl ether, butyronitrile, acetonitrile, chlorobenzene, including any combination thereof.

According to some embodiments, the core further comprises at least one additional ingredient selected from an active ingredient, a cell-nutrition ingredient or both.

According to some embodiments, a viability of the plurality of cells within the polymeric fiber is maintained for at least 2 weeks.

As used herein, the term “matrix” refers to one or more porous layers of polymeric fibers randomly, and/or under certain order or control, distributed therewithin. In some embodiments, the terms “fibrous material” and “matrix” are used herein interchangeably. Matrix may further include any materials incorporated within and/or interposed between the layers. In some embodiments, the matrix comprises randomly oriented polymeric fibers. In some embodiments, each polymeric fiber within the matrix is in contact with at least one additional polymeric fiber. In some embodiments, the polymeric fibers are randomly distributed within the matrix, so obtain a three-dimensional mesh structure comprising a void space between the fibers. In some embodiments, the polymeric fibers are randomly distributed within the matrix thus forming a plurality of pores (or void space).

In some embodiments, the terms “layer”, and “film” are used herein interchangeably, and refer to a material having a substantially uniform-thickness. In some embodiments, the term “layer” refers to a substantially homogeneous material characterized by a substantially the same chemical composition and/or substantially the same three-dimensional structure. In some embodiments, layer is characterized by a homogenous or uniform feature within the entire layer, wherein the feature is selected from spatial distribution of the polymeric fibers, fiber thickness, pore size, porosity, thickness, including any range between. In some embodiments, the term “entire layer” refers to at least 50%, at least 60%, at least 70%, at least 80%, at least 90%, at least 95%, at least 97% of the surface and/or volume of the layer, including any range between.

The terms “hydrophilic polymer” and “water-soluble polymer” are used herein interchangeably. The terms “hydrophobic polymer” and “water insoluble polymer” are used herein interchangeably. The terms “porous shell” and “shell” are used herein interchangeably and refer to the outer portion of the polymeric fiber of the invention facing the ambient and enclosing or encapsulating the core.

The “hydrophilic core” and “core” are used herein interchangeably and refer to the inner portion of the polymeric fiber of the invention in direct contact with and encapsulated by the shell.

In some embodiments, the fibrous material is in a form of a fibrous mat. In some embodiments, the fibrous mat is in a form of a non-woven layer. In some embodiments, the fibrous mat is in a form of a layer characterized by a relatively uniform thickness, a uniform spatial distribution of the polymeric fibers, or both. In some embodiments, the polymeric fibers are electrospun fibers (e.g. electrospun microfibers). In some embodiments, the polymeric fibers have a cylindrical shape, or a tub-like shape (e.g. a hollow tube).

In some embodiments, the cells and/or spores are encapsulated by the polymeric shell of the polymeric fiber, wherein at least 70%, at least 80%, at least 85%, or between 60 and 95%, between 70 and 100%, between 70 and 95% of the initial amount of the encapsulated cells and/or spores retain viability for at least 2 weeks, at least 1 month, 1 year or even more.

### Polymeric Fibers

In one aspect, there is provided a polymeric fiber (e.g. electrospun microfiber) comprising a porous shell encapsulating, enclosing and/or in direct contact with a hydrophilic core. In some embodiments, the polymeric fibers of the invention are electrospun fibers. In some embodiments, the polymeric fibers of the invention are composed essentially of electrospun fibers (e.g. electrospun microfibers). In some embodiments, the porous shell is as described herein, and the hydrophilic core comprises a plurality of cells and/or a plurality of spores and a water-soluble material, wherein the water-soluble material constitutes up to 50%, up to 40%, up to 30%, up to 20%, up to 15%, up to 10%, up to 5%, up to 3%, up to 2%, up to 1%, up to 0.5%, up to 0.2%, between about 0.1 and about 20%, between about 0.2 and about 25%, between about 0.2 and about 30%, between 1 and 20%, between 2 and 20%, between 5 and 20%, between 10 and 20%, between 5 and 15%, between 5 and 10% by dry weight of the polymeric fiber, including any range between.

In some embodiments, the water-soluble material is a thickener. In some embodiments, the water-soluble material is or comprises a water-soluble polymer. In some embodiments, the water-soluble material is or comprises a mono-saccharide, a di-saccharide, an oligosaccharide or any combination thereof. In some embodiments, the water-soluble material comprises a sugar (e.g. a sugar as disclosed herein), and a hydrophilic polymer as disclosed herein, wherein the w/w concentration of the water-soluble polymer constitutes up to 20%, up to 15%, up to 10%, up to 5%, up to 3%, up to 2%, up to 1%, up to 0.5%, by dry weight of the hydrophilic core, including any range between. In some embodiments, the water-soluble polymer is a thickener or filler. In some embodiments, the porous shell of the polymeric fiber is a solid (or solid porous she)ll; and the hydrophilic core is in a solid or a semi-solid state.

In some embodiments, the water-soluble material comprises a single specie of the water-soluble polymer, and/or a single species of sugar. In some embodiments, the water-soluble material comprises a plurality of chemically distinct water soluble polymer species, and/or a plurality of In some embodiments, the water-soluble material comprises a plurality of chemically distinct sugar species.

In some embodiments, the water-soluble polymer is capable of modifying the viscosity of the electrospinning core solution, so as to obtain a predetermined viscosity sufficient for forming the electrospun fibers of the invention. In some embodiments, the water-soluble polymer comprises a water-soluble polysaccharide, polyalkyleneoxide (e.g. polyethyleneglycol (PEG), polypropylene glycol (PPG)), a polyol, a polyvinyl alcohol (PVA), a polyether, polyvinyl pyrrolidone (PVP) or any combination thereof. In some embodiments, the water-soluble polymer is or comprises a polysaccharide. Numerous water-soluble polysaccharides suitable for implementation within the core of the polymeric electrospun fiber are known in the art. Exemplary water-soluble polysaccharides and additional polymers suitable for the manufacturing of the polymeric electrospun fiber are listed in WO2008/041183 which is incorporated herein by reference in its entirety.

Non-limiting examples of water-soluble polysaccharides include but are not a water-soluble derivative of cellulose (e.g., carboxylated cellulose such as HPMC), alginic acid, hyaluronic acid, chitosan, a water-soluble gum (e.g., guar gum, locust bean gum, gellan gum, Xanthan gum, Acacia gum, Gum Arabic), a carrageenan (e.g. lambda-carrageenan) including any copolymer, or any combination thereof.

In some embodiments, the core further comprises an active ingredient (e.g. a pharmaceutically active agent, a cosmeceutical active agent, a nutraceutical or any combination thereof). In some embodiments, the core further comprises a cell-nutrition ingredient (e.g. dried culture medium constituents such as growth factors, carbohydrate, co-factors, etc.).

In some embodiments, the core of the polymeric fiber of the invention consists essentially of the water-soluble material and the plurality of cells, wherein water soluble material comprises or consists essentially of the water-soluble polymer and the sugar. In some embodiments, between 70 and 100%, between 70 and 95%, between 80 and 99%, between 90 and 99%, between about 90 and 100% of the water-soluble material within the polymeric fiber of the invention is composed of the water-soluble polymer and the sugar.

In some embodiments, the core is a homogenous core comprising a matrix composed of intertwined polymeric chains of the water-soluble polymer and the sugar. In some embodiments, the sugar and the water-soluble polymer are homogenously mixed or distributed within the core. In some embodiments, at least 10%, at least 20%, at least 30%, at least 40%, at least 50%, at least 70%, at least 90%, at least 95% of the polymeric fiber's volume is filled with the core constituents, including any range between. In some embodiments, at least 10%, at least 20%, at least 30%, at least 40%, at least 50%, at least 70%, at least 90%, at least 95% of the core is void, including any range between. In some embodiments, the polymeric fiber with at least 30% or more of the core being a void space, refers to herein as a hollow tube-like structure.

In another aspect, there is provided a polymeric fiber (e.g. electrospun microfiber) comprising or encapsulating a plurality of cells, a plurality of spores or both; wherein the polymeric fiber comprises a porous hydrophobic shell and a hydrophilic core, and wherein the hydrophilic core comprises or encapsulates the plurality of cells and the water-soluble material; and wherein the porous hydrophobic shell comprises or is composed essentially of a water-insoluble polymeric material. In some embodiments, the plurality of cells comprise one or more microorganisms. In some embodiments, the plurality of cells are dormant and/or viable cells. In some embodiments, the plurality of cells are distinct cell species, or are the same cell specie. In some embodiments, the plurality of cells comprise one or more microbial cell, one or more microbial spores, one or more microorganism, or any combination thereof.

In some embodiments, the polymeric fiber comprises a solid porous shell and a solid or a semi-solid core. In some embodiments, the polymeric fiber comprising the plurality of cells has a cylinder-like structure. In some embodiments, the core of the cylinder-like structured polymeric fiber comprises less than 90%, less than 80%, less than 70%, less than 60%, less than 50%, less than 40%, less than 30%, less than 20%, less than 10%, less than 5%, or between 5 and 50%, between 10 and 90%, between 5 and 50%, between 10 and 70%, between 10 and 30% void space, relative to the entire volume of the core, including any range between. In some embodiments, the polymeric fiber comprising or encapsulating a plurality of cells is referred to herein as the polymeric fiber of the invention.

In some embodiments, the polymeric fibers of the invention are characterized by an average cross-section between 1 and 500 um, between 1 and 10 um, between 10 and 100 um, between 10 and 50 um, between 50 and 100 um, between 50 and 300 um, between 50 and 400 um, between 50 and 500 um, between 10 and 500 um, between 10 and 300 um, between 100 and 500 um, between 10 and 300 um, between 10 and 400 um, between 10 and 150 um, between 10 and 200 um, between 10 and 350 um, between 100 and 300 um, between 300 and 500 um, including any range between.

In some embodiments, the polymeric fibers of the invention are characterized by an average cross-section between 10 and 500 um, between 10 and 100 um, between 10 and 200 um, between 10 and 150 um, between 10 and 300 um, including any range between, wherein the porous shell of the polymeric fiber is a porous solid shell composed essentially of a water-insoluble polymer and optionally a water soluble polymer (as disclosed hereinbelow); the hydrophilic core of the polymeric fiber is composed essentially of the plurality of cells and the water soluble material; and wherein the water soluble material in the core of the polymeric fiber is composed essentially of a water-soluble polymer and one or more sugar.

In some embodiments, the polymeric fibers of the invention are characterized by an average cross-section between 10 and 500 um, between 10 and 100 um, between 10 and 200 um, between 10 and 150 um, between 10 and 300 um, including any range between, wherein the porous shell of the polymeric fiber is a porous solid shell composed essentially of a water-insoluble polymer (e.g. cellulose, a water-insoluble cellulose derivative, or PVDF-HFP), and optionally a water soluble polymer (as disclosed hereinbelow); the hydrophilic core of the polymeric fiber is composed essentially of the plurality of cells and the water soluble material consisting essentially of a sugar and the water-soluble polymer disclosed herein (e.g. a water soluble gum, polyalkylenoxide, etc.); and wherein the water soluble material in the core of the polymeric fiber is composed essentially of a water-soluble polymer and one or more sugar.

In some embodiments, a w/w concentration of the water-soluble polymer within the polymeric fibers or within the fibrous material of the invention is between 0.1 and 20%, between 0.5 and 20%, between 0.5 and 15%, between 0.5 and 10%, between 1 and 20%, between land 15%, between 1 and 10%, between 1 and 5%, between 5 and 20% m between 5 and 15%, including any range between.

In some embodiments, a w/w concentration of the water-insoluble polymer within the polymeric fibers or within the fibrous material of the invention is between 2 and 30%, between 5 and 30%, between 10 and 30%, between 10 and 20%, between 2 and 20%, between 5 and 15%, between 5 and 10%, between 5 and 20%, including any range between.

In some embodiments, the polymeric fibers are characterized by an average length from about 0.1 millimeter (mm) to about 20 centimeter (cm) or more, e.g., from about 1-20 cm, e.g., from about 5-10 cm, between 0.1 and 200 mm, between 0.1 and 1 mm, between 1 and 10 mm, between 10 and 50 mm, between 50 and 100 mm, between 10 and 200 mm, between 50 and 200 mm, between 100 and 200 mm, including any range between.

In some embodiments, the polymeric fibers are characterized by an average aspect ratio (e.g., a ratio between the length and the cross-section of the polymeric fiber) of at least 10, at least 100, at least 1000, at least 10,000, at least 50,000 including any range between.

In some embodiments the polymeric fibers are characterized by an average aspect ratio (e.g. a ratio between the length and the cross-section of the polymeric fiber) representing a theoretical 2D structure.

In some embodiments, the porous shell of the polymeric fibers comprises a plurality of pores. In some embodiments, the pore size and pore density per surface unit of the polymeric fiber (or porosity) is sufficient to for maintaining viability of the cells encapsulated within the polymeric fiber. In some embodiments, the pore size and pore density per surface unit of the polymeric fiber (or porosity) is sufficient for supporting activation of the cells encapsulated within the polymeric fiber, upon contacting thereof with an activation solution. In some embodiments, pores size sufficient for supporting activation is a pore size capable of supporting a mass transfer of cell nutrients (e.g. from the ambient to the core, and/or from the core to the ambient) sufficient for inducing and/or maintaining cell activity (e.g. essential biological activity, such as cell metabolism). The term “mass transfer”, as used herein refers to a net movement of a liquid composition comprising cell nutrients dissolved or dispersed therewithin, wherein the net movement is from the ambient solution to the core of the polymeric fibers, or from the core of the polymeric fibers to the ambient solution. In some embodiments, the viability of the cells predetermines the shelf life of the article of the invention. In some embodiments, the viability of the cells is maintained if at least 50%, at least 60%, at least 70%, between 50 and 100% m between 50 and 90%, between 70 and 100%, between 70 and 95% of the initial cell loading are viable (cell loading is defined inter alia as CFU number per gram of the article, or CFU number per cm² of the article). In some embodiments, supporting activation is so as to obtain activation of at least 50%, at least 60%, at least 70%, between 50 and 100%, between 50 and 90%, between 70 and 100%, between 70 and 95% of the initial cell loading, including any range between.

In some embodiments, porosity sufficient for supporting activation of the cells encapsulated within the polymeric fiber is at least 50%, at least 60%, at least 70%, between about 60 and 90%, between about 70 and about 90%, between about 70 and about 95%, including any range between. The term “porosity” is used herein refers to an average value of the composition comprising the plurality of polymeric fibers. Porosity can be determined based on the microscopic images of the fibers, such as by SEM.

In some embodiments, the pores within the porous shell of the polymeric fiber are characterized by an average pore size between 10 and 500 nm, between 10 and 300 nm, between 100 and 500 nm, between 20 and 80 nm, 80 and 120 nm, between 50 and 200 nm, between 80 and 200 nm, between 150 and 200 nm, between 50 and 500 nm, including any range between.

In some embodiments, the polymeric fibers of the invention are characterized by (i) an average pore size between 30 and 300 nm, between 30 and 400 nm, between 30 and 500 nm, between 40 and 400 nm, between 40 and 300 nm, between 50 and 300 nm, between 50 and 400 nm, between 50 and 500 nm, between about 50 and about 300 nm, including any range between; and by (ii) porosity of at least 50%, at least 60%, at least 70%, between about 60 and 90%, between about 70 and about 90%, between about 70 and about 95%, including any range between. In some embodiments, the polymeric fibers of the invention suitable for supporting activation of the cells encapsulated therewithin are characterized by an average pore size between about 50 and about 300 nm; and by a porosity of at least about 70%.

In some embodiments, the polymeric fibers are characterized by a plurality of pores, as described hereinabove, and are further characterized by an opening (e.g. incision) having an average size ranging between 1 and 10 um, between 0.5 and 10 um, between 1 and 100 um, between 1 and 2 um, between 1 and 10 um, between 1 and 2 um, between 2 and 5 um, between 5 and 10 um, including any range between. A skilled artisan will appreciate that the average pore size may be calculated based on SEM micrographs of the matrix.

In some embodiments, the polymeric fibers are characterized by an average pore size of about 1 micron.

In some embodiments, the shell of the polymeric fiber is a hydrophobic shell comprising a water-insoluble polymer. In some embodiments, the water-insoluble polymer is characterized by water solubility of at most 0.5 g/L, at most 0.1 g/L, at most 0.05 g/L, at most 0.01 g/L, at most 0.005 g/L, at most 0.001 g/L, or less including any range between.

In some embodiments, the water-insoluble polymer is characterized by solubility of at least 5 g/L, at least 10 g/L, at least 20 g/L, at least 50 g/L, at least 100 g/L within a water immiscible organic solvent, including any range between. In some embodiments, the water-insoluble polymer is a water dispersible polymer.

In some embodiments, the water-insoluble polymer is characterized by solubility of at least 5 g/L, at least 10 g/L, at least 20 g/L, at least 50 g/L, at least 100 g/L within a water-miscible or water-immiscible organic solvent (e.g. when measured at a temperature between 1° and 30° C., between 1° and 50° C., or between 0 and 90° C.). In some embodiments, the water-insoluble polymer is characterized by solubility of at least 5 g/L, at least 10 g/L, at least 20 g/L, at least 50 g/L, at least 100 g/L within a solvent selected from THF, DMF, acetone, DMAC, chloroform, DMSO, DCM, NMP, TCE, TFE, butanol, methanol, ethanol, HFP, Isopropanol, Ethyl acetate, Ethanolamine, pentane, ethylene glycol, Diethyl ether, butyronitrile, acetonitrile, chlorobenzene, including any combination thereof.

In some embodiments, the water-insoluble polymer comprises a fluoropolymer including any copolymer thereof. In some embodiments, the hydrophobic polymer is or comprises PVDF-HFP (Poly(vinylidene fluoride-hexafluoropropylene). In some embodiments, the water-insoluble polymer comprises or consist essentially of cellulose, and/or a water-insoluble cellulose derivative.

In some embodiments, the water-insoluble polymer is selected from cellulose, a fluoropolymer (e.g. PVDF-HFP), PVP, PCL, PLA, PGA, polystyrene, PES, an acrylate polymer, a water insoluble polysaccharide, a water insoluble cellulose derivative, including any combination and any co-polymer thereof. In some embodiments, the water insoluble cellulose derivative include but are not limited to: carboxyalkylated cellulose, (e.g. carboxy methyl cellulose or CMC, hydroxypropyl methyl cellulose), alkylated cellulose (e.g. Me-cellulose, ethyl cellulose, ethyl methyl cellulose), hydroxyalkylated cellulose (e.g. hydroxy ethyl cellulose, hydroxy propyl cellulose), alkoxylated cellulose, acylated cellulose (e.g. cellulose acetate, cellulose acetate phthalate, including any combination and any copolymer thereof.

In some embodiments, the shell comprises up to 90%, up to 88%, up to 87%, up to 86% w/w of the water-insoluble polymer, by dry weight of the shell. In some embodiments, the shell comprises between 50 and 90%, between 50 and 80%, between 70 and 90%, between 60 and 70%, between 70 and 86%, between 60 and 86%, between 80 and 90%, between 80 and 85%, between 80 and 88%, between 80 and 86%, between 80 and 83%, between 83 and 90%, between 83 and 86%, between 85 and 88% of the water-insoluble polymer, including any range between.

In some embodiments, the shell further comprises up 20%, up 15%, up to 13%, up to 10%, up to 8%, up to 5% or between 5 and 20%, between 5 and 15% by weight of a water-soluble polymer, including any range between. In some embodiments, the water-soluble polymer is as described hereinabove. In some embodiments, the water-soluble polymer is or comprises PVP, or a polyether (e.g. a polyalkyleneoxide such as polyethyleneglycol (PEG), polypropylene glycol (PPG)). In some embodiments, the shell comprises or consists essentially of a mixture of PVDF-HFP (as a hydrophobic polymer) and PVP (as a water-soluble polymer), wherein the w/w concentration of each of the PVDF-HFP and PVP is as described herein.

In some embodiments, an average molecular weight of the water-insoluble polymer is between 100.000 and 2.000.000 Da, between 100.000 and 1.000.000 Da, between 100.000 and 300.000 Da, between 300.000 and 500.000 Da, between 100.000 and 500.000 Da, between 200.000 and 800.000 Da, between 300.000 and 800.000 Da, between 500.000 and 800.000 Da, between 1.000.000 and 2.000.000 Da, between 1.000.000 and 1.500.000 Da, between 1.500.000 and 2.000.000 Da, between 500.000 and 800.000 Da, between 500.000 and 800.000 Da, including any range between.

In some embodiments, an average molecular weight of PVDF-HFP is between 100.000 and 800.000 Da, between 100.000 and 300.000 Da, between 300.000 and 500.000 Da, between 100.000 and 500.000 Da, between 200.000 and 800.000 Da, between 300.000 and 800.000 Da, between 500.000 and 800.000 Da, including any range between.

In some embodiments, an average molecular weight of PVP is between 500.000 and 2.000.000 Da, between 500.000 and 1.000.000 Da, between 1.000.000 and 2.000.000 Da, between 1.000.000 and 1.500.000 Da, between 1.500.000 and 2.000.000 Da, between 500.000 and 800.000 Da, between 500.000 and 800.000 Da, including any range between.

In some embodiments, the term “average molecular weight” refers to a weight average molecular weight (Mw), which is well-known in the art. In some embodiments, the term “average molecular weight” refers to a number average molecular weight (Mn). In some embodiments, the term “Mn” generally refers to a molecular weight measurement that is calculated by dividing the total weight of all the polymer molecules in a sample with the total number of polymer molecules in the sample.

In some embodiments, the shell is characterized by a thickness between 10 nm and 10 um, between 50 and 500 nm, between 50 nm and 1 um, between 50 nm and 10 um, between 50 and 100 nm, between 100 and 200 nm, between 100 and 500 nm, between 100 and 1000 nm, between 500 and 700 nm, between 500 and 1000 nm, between 100 nm and 5 um, between 100 nm and 1 um, between 100 nm and 10 um, including any range between.

In some embodiments, the shell further comprises a trace amount of any one of water, or a water-immiscible organic solvent, or both.

In some embodiments, a w/w ratio between the shell and the core within the polymeric fiber is between 50:1 and 1:50, between 5:1 and 1:50, between 1:1 and 1:50, between 1:5 and 1:50, between 1:5 and 1:10, between 1:10 and 1:50, between 1:10 and 1:20, between 1:10 and 1:30, between 1:20 and 1:50, between 1:30 and 1:50, including any range between.

In some embodiments, the core is in a solid or a semi-solid state. In some embodiments, the core stably encapsulates the cells. In some embodiments, the cells are stably encapsulated within the core. In some embodiments, the cells are stably bound to the core constituents or encapsulated within the core constituents. In some embodiments, the core is substantially devoid of pores. In some embodiments, the core is substantially water vapor and/or gas impermeable. In some embodiments, the core comprises a voluminous filler. In some embodiments, the core comprises one or more materials capable of expanding during the electrospinning process, thereby providing a mechanical support for the entire polymeric fiber. In some embodiments, the core provides or enhances the mechanical stability of the fiber. In some embodiments, the core is in a form of a gel, or a glassy material.

In some embodiments, the core is in a glass state at a temperature between 1° and 30° C., between 1° and 40° C., between 1° and 50° C., between 1° and 60° C., or more including any range between. In some embodiments, the core comprises a plurality of cells, the sugar and the water-soluble polymer. In some embodiments, the plurality of cells constitute up to 90%, up to 80%, up to 70%, up to 60%, up to 50%, up to 30%, by dry weight of the core, including any range between. In some embodiments, the plurality of cells constitute up to 90%, up to 80%, up to 70%, up to 60%, up to 50%, up to 30%, up to 20%, by dry weight of the polymeric fiber of the invention.

In some embodiments, the water-soluble polymer is characterized by water solubility of at least 1 g/L, at least 5 g/L, at least 10 g/L, at least 20 g/L, at least 50 g/L, at least 100 g/L, including any range between. In some embodiments, the water-soluble polymer is characterized by solubility of at least 5 g/L, at least 10 g/L, at least 20 g/L, at least 50 g/L, at least 100 g/L within an aqueous solvent (e.g., an aqueous buffer, saline or water; when measured at a temperature between 1° and 30° C., between 1° and 50° C., or between 0 and 90° C.).

In some embodiments, the water-soluble polymer comprises a single polymeric specie. In some embodiments, the water-soluble polymer comprises a plurality of distinct polymeric species. In some embodiments, the water-soluble polymer is or comprises a water-soluble polysaccharide. In some embodiments, the water-soluble polysaccharide is a water-soluble gum. In some embodiments, the water-soluble gum is or comprises a carrageenan (e.g., lambda-carrageenan). In some embodiments, the water-soluble polysaccharide is or comprises a carrageenan (e.g. lambda-carrageenan). In some embodiments, the water-soluble polymer consists essentially of a water-soluble gum.

In some embodiments, the water-soluble polymer and/or the sugar is substantially non-hygroscopic. In some embodiments, the water-soluble polymer and/or the sugar is characterized by a significantly lower hygroscopy than the hygroscopy of glycerol. In some embodiments, the water-soluble material as disclosed herein is characterized by a significantly lower hygroscopy than the hygroscopy of glycerol (e.g. 1.5, 2, 3, 5, or 10 times lower hygroscopy, including any range between).

In some embodiments, the sugar comprises one or more of a di-saccharide, a mono-saccharide, an oligosaccharide, or any combination thereof.

Non-limiting examples of di-saccharides and mono-saccharides include but are not limited to D- and/or L-sugar such as sucrose, maltose, fructose, glucose, galactose, isomaltulose, trehalose, psicose, tagatose, and sorbose including any combination thereof.

In some embodiments, the sugar comprises a first sugar (e.g. sucrose) and a second sugar (e.g. maltose). In some embodiments, a w/w ratio between the first sugar and the second sugar within the core (and/or within the fiber) is between 1:1 and 20:1, between 1:1 and 5:1, between 5:1 and 10:1, between 10:1 and 15:1, between 15:1 and 20:1, including any range between.

In some embodiments, the sugar weight content within the core is at least 50%, at least 60%, at least 70%, at least 80%, at least 90%, at least 95% w/w of the dry weight of the core, including any range between. In some embodiments, the sugar weight content within the fiber is at least 50%, at least 60%, at least 70%, at least 80%, at least 90%, at least 95% w/w of the dry weight of the fiber, including any range between.

In some embodiments, the water-soluble polymer constitutes up to 20%, up to 15%, up to 10%, up to 5%, up to 3%, up to 2%, up to 1%, up to 0.5%, by dry weight of the core, including any range between. In some embodiments, the water-soluble polymer constitutes up to 10%, up to 7%, up to 5%, up to 3%, up to 2%, up to 1%, up to 0.5%, by dry weight of the core, including any range between. In some embodiments, the water-soluble polymer constitutes up to up to 5%, up to 3%, up to 2%, up to 1%, up to 0.5%, by dry weight of the core, including any range between. In some embodiments, the water-soluble polymer constitutes at least 0.1%, at least 0.3%, at least 0.5%, at least 0.7%, at least 1%, at least 2% by dry weight of the core, including any range between.

In some embodiments, the water-soluble polymer is or comprises lambda-carrageenan. In some embodiments, the water-soluble polymer consists essentially of lambda-carrageenan. The inventors surprisingly found that lambda-carrageenan is preferential for obtaining a polymeric fiber stably encapsulating the dormant cells, wherein the dormant cell retained their viability for a prolonged time period of at least 3 months, or at least 6 months. Thus, it has been surprisingly found that the mat of the invention comprising sucrose and maltose as the sugar, and lambda-carrageenan as the water-soluble polymer of the core is characterized by prolonged shelf-life (e.g. at least 1 month, at least 3 months, at least 6 months, at least 9 months, at least 12 months or more) when stored under regular storage conditions as described herein.

In some embodiments, the core further comprises an active ingredient (e.g. a pharmaceutically active agent, a cosmeceutically active agent, a nutraceutical, a cell-nutrition constituent or any combination thereof).

In some embodiments, the polymeric fiber comprises between 40 and 98%, between 50 and 98%, between 70 and 98%, between 60 and 98%, between 70 and 95%, between 60 and 95%, between 80 and 95%, between 40 and 90%, between 50 and 90%, between 80 and 95%, between 80 and 98%, of the plurality of cells and/or a plurality of spores, dry weight per weight (w/w) of the polymeric fiber including any range between.

In some embodiments, the polymeric fibers of the invention are electrospun microfibers; wherein at least 85%, at least 90%, at least 95%, at least 99%, or between 85 and 95%, between 85 and 100%, between 90 and 10%, between 95 and 99% from the dry weight of the polymeric fibers of the invention consist of the water-soluble material and the plurality of cells forming the hydrophilic core encapsulated by the porous hydrophobic shell; wherein the porous hydrophobic shell consists essentially of the water-insoluble polymer and optionally of the water-soluble polymer; and wherein the water-soluble material consists essentially of the water-soluble polymer and sugar; and wherein the water-soluble polymer, the water-insoluble polymer and the sugar are as described hereinabove.

In some embodiments, at least 85%, at least 90%, at least 95%, at least 99%, or between 85 and 95%, between 85 and 100%, between 90 and 10%, between 95 and 99% from the dry weight of the polymeric fibers of the invention consist of (i) one or more water-soluble polymer(s) such as PEG and/or a water-soluble gum; (ii) one or more sugar(s); (iii) a water-insoluble polymer, such as cellulose or a water-insoluble cellulose derivative, and optionally a water-soluble polymer; and (iv) a plurality of cells encapsulated within the polymeric fiber; wherein (i) and (ii) form the water soluble material of the core, and wherein (iii) forms the porous shell of the polymeric fiber.

### Fibrous Material

In one aspect of the invention, there is provided a fibrous material comprising electrospun fibers disclosed herein, wherein each of the electrospun fibers comprises a hydrophobic shell as disclosed herein, and a hydrophilic core comprising the water-soluble material and a plurality of cells, as disclosed herein. In some embodiments, the hydrophilic core is in a form of a hollow tube. In some embodiments, the electrospun fibers of the fibrous material are substantially devoid of dead cells. In some embodiments, the fibrous material is a dry fibrous material characterized by a moisture content of less than 10%, less than 5%, less than 3%, less than 1%, less than 0.5%, less than 0.1%, less than 0.05%, less than 0.01%, including any range between. In some embodiments, the fibrous material is a dry mat.

In another aspect of the invention, there is provided herein a fibrous material comprising the polymeric fibers of the invention, wherein the polymeric fibers comprising a plurality of cells, one or more microorganism(s), and/or spores encapsulated therewithin, wherein the cells or spores are dormant and/or viable. In some embodiments, the fibrous mater is in a form of a fibrous mat comprising one or more non-woven uniform layer(s) or matrix.

In some embodiments, the fibrous material is a porous matrix, characterized by an average porosity of about 10%, 20%, about 50%, about 70%, about 90%, between 50 and 99%, between 60 and 90% or more, including any range between.

In some embodiments, the fibrous material is characterized by an average pore size ranging between 10 and 400 um, between 10 and 50 um, between 50 and 80 um, between 80 and 100 um, between 100 and 150 um, between 150 and 200 um, between 150 and 300 um, between 100 and 400 um, between 150 and 400 um, between 200 and 400 um, or greater than 100 um, including any range between. In some embodiments, the fibrous material is characterized by an average pore size of 1 micron or more. In some embodiments, the fibrous material is characterized by an average pore size of less than 10 um. In some embodiments, the pore size of the fibrous material refers to a void space between the polymeric fibers within the matrix.

A skilled artisan will appreciate that the average pore size may be calculated based on SEM micrographs of the matrix.

In some embodiments, the fibrous material is characterized by an average thickness of between 10 and 10000 um, between 10 and 2000 um, between 10 and 50 um, between 50 and 100 um, between 100 and 200 um, between 100 and 500 um, between 500 and 1000 um, between 100 and 2000 um, between 200 and 2000 um, between 500 and 2000 um, between 1000 and 2000 um, between 2000 and 10000 um, between 2000 and 5000 um, between 2000 and 7000 um, including any range between.

In some embodiments, the fibrous material is substantially dry. In some embodiments, the mat is substantially devoid of moisture. In some embodiments, the mat is wet. In some embodiments, the fibrous material is moist. In some embodiments, the moisture content of the mat is less than 10%, less than 5%, less than 3%, less than 1%, less than 0.5%, less than 0.1%, less than 0.05%, less than 0.01%, including any range between. In some embodiments, the fibrous material of the invention comprises trace amounts of water and/or organic solvents.

In some embodiments, the fibrous material of the invention in a dry state is characterized by a prolonged shelf life, when stored under appropriate storage conditions as described herein. In some embodiments, the shelf life refers to the ability of the fibrous material to substantially retain viability of the dormant cells, as described herein. In some embodiments, viability of dormant cells is the ability of the cells to proliferate and/or secrete metabolites. In some embodiments, the fibrous material includes live-active (not dormant) cells. In some embodiments, the fibrous material includes a plurality of live-active (not dormant) cells. In some embodiments, the fibrous material is substantially devoid of dead cells (e.g., not more than 30%, 20%, or 10% dead cells, relative to the total cell count within the fibrous material.

In some embodiments, the fibrous material comprises a plurality of dormant cells encapsulated with the core of the polymeric fibers. In some embodiments, a dry weight per weight (w/w) concentration of the encapsulated dormant cells within the mat is up to 98%, up to 95%, up to 93%, up to 90%, up to 80%, up to 70%, up to 60%, up to 50%, up to 40%, relative to the dry weight of the fibrous material. A skilled artisan will appreciate that the cell loading in the fibrous material may vary, and is predetermined by the desired properties of the fibrous material. Usually, it is desirable to have a material with the highest possible loading of the encapsulated cells/spore, and/or microorganism(s).

In some embodiments, a dry weight per weight (w/w) concentration of the cells within the fibrous material is between about 10 and about 98%, between about 10 and about 95%, between about 10 and about 93%, between about 10 and about 90%, between about 10 and about 80%, between 10 and 30%, between 20 and 30%, between 30 and 40%, between 30 and 80%, between 30 and 50%, between 50 and 80%, between 40 and 80%, between 40 and 60%, between 60 and 80%, including any range between.

In some embodiments, the fibrous material of the invention is characterized by a cell loading (e.g. CFU of active cells per area or volume of the mat) sufficient for secreting a cosmeceutically effective amount of the metabolite (or cosmeceutical active ingredient). In some embodiments, the mat of the invention is characterized by a cell loading sufficient for supplementing a skin of the subject with the effective amount of the metabolite (e.g. upon activation of the dormant cells with the with the activation solution, as described herein).

In some embodiments, the cell loading is at least 10E3, at least 10E5, at least 10E7, at least 10E10, at least 10E13, between 10E5 and 10E15, between 10E7 and 10E15, between 10E10 and 10E15, between 10E6 and 10E15, between 10E8 and 10E15, between 10E5 and 10E13, between 10E10 and 10E13 CFU/cm², including any range between.

As used herein the term “cell” refers to a eukaryotic or prokaryotic cell.

According to some embodiments of the invention, the cell comprises a cell wall. Non-limiting examples of cells which comprise a cell wall and which can be encapsulated within the polymeric fibers of the invention include plant cells, bacteria (e.g., Gram positive and Gram-negative bacteria), archaea, protozoa, fungi, yeast and algae, including any spore thereof and/or any combination thereof.

According to some embodiments of the invention, the cell comprises a cosmeceutically-acceptable cell (e.g., a cosmeceutically pure bacteria and/or bacteria approved for cosmetic use). In some embodiments, the cell comprises a probiotic bacteria. In some embodiments, the cell comprises a single bacterial specie. In some embodiments, the cell comprises a plurality of bacterial species. In some embodiments, the cell comprises a microbial cell, a microbial spore, or both. In some embodiments, the cell is or comprises a plant cell. In some embodiments, the cell is or comprises an animal cell (e.g. a mammalian cell). In some embodiments, the cell comprises bacteria of genus Lactiplantibacillus, including any sub-species thereof. In some embodiments, the cell comprises Lactiplantibacillus plantarum. In some embodiments, the cell comprises bacteria of genus Lactobacillus, including any sub-species thereof.

In some embodiments, the cell comprises a microorganism capable of secreting a metabolite (e.g., wherein the cell is in the activated state). In some embodiments, the metabolite is a cosmeceutically active ingredient. In some embodiments, the cell is or comprises a dormant cell, wherein the dormant cell is a probiotic microbe and/or a spore thereof capable of secreting a metabolite upon activating thereof (e.g. by exposing the cell to the activating composition as disclosed herein).

In some embodiments, the cell is an isolated cell (e.g. purified of the growth medium, debris, nutrients, etc.). In some embodiments, the cell is an isolated bacteria. In some embodiments, the cell is devoid of a human pathogen, and/or toxin. In some embodiments, the cell is characterized by a purity (e.g. a cell-culture purity) of at least 90%, at least 95%, at least 97%, at least 99%, at least 99.9%, at least 99.99%, including any range between.

In some embodiments, at least 60%, at least 70%, at least 80%, at least 90%, at least 95%, at least 97%, at least 99% of the total number (e.g. CFU) of the cells within the mat are encapsulated or located within the core of the polymeric fibers. Without being bound to any specific theory, it is postulated that the dormant cells are primarily encapsulated within the fibers, so that only a minor portion (e.g. at most 30%, at most 20%, at most 10%, at most 5% including any range between) of the dormant cells is optionally located outside the fiber's core.

In some embodiments, at least 60%, at least 70%, at least 80%, at least 90%, at least 95%, at least 97%, at least 99% of the total number (e.g. CFU) of the cells within the mat remain encapsulated or located within the core of the polymeric fibers, upon wetting of the mat with a liquid (e.g. activation solution). Without being bound to any theory, it is postulated that upon wetting of the mat the metabolites can freely diffuse into the liquid (located at the outer portion of the wet mat), whereas the encapsulated bacteria substantially remain within the fiber's core. It should be further apparent that due to the mass transfer enabled by a porous fiber shell, at least a portion of the metabolites can reach the skin of the subject.

In some embodiments, the cells encapsulated within the polymeric fibers are substantially viable. In some embodiments, at least 50%, at least 60%, at least 70%, at least 80%, at least 90%, at least 95%, at least 97%, at least 99%, at least 99.9% of the cells encapsulated within the polymeric fibers are viable. In some embodiments, the term “viable” as used herein, encompasses being capable of: replicating a genome or DNA, cell proliferation or replication, RNA synthesis, protein translation, fermentation or any equivalent energy production process, secretion of an active compounds (e.g. metabolite or a cell metabolite such as disclosed herein), or any combination thereof. In some embodiments, the term “viable” as used herein refers to the cell in an active state. In some embodiments, the term “viable” further encompass the capability of the cell to undergo transformation from a dormant state into an active state, such as upon contacting thereof with an activation composition, as disclosed herein. In some embodiments, the transformation from a dormant state into an active state also refers to herein as the activation of the dormant cells.

In some embodiments, the cell encapsulated within the polymeric fibers are in a dormant state. In some embodiments, the cell encapsulated within the polymeric fibers are capable of undergo transformation from a dormant state into an active state. In some embodiments, the cell in the active state is capable of performing any cellular biochemical process known in the art. In some embodiments, the cell in the active state is capable of: replicating a genome or DNA, cell proliferation or replication, RNA synthesis, protein translation, fermentation or any equivalent energy production process. In some embodiments, the cell in the active state is capable of synthesizing and/or secretion of an active compounds (e.g. a metabolite such as a cosmeceutically active ingredient). In some embodiments, the cell in the active state is characterized by synthesis and secretion of an active compounds. One skilled in the art would appreciate, that the cell viability can be determined and/or monitored by analyzing the concentration of one or more metabolites within the growth medium. In a non-limiting exemplary embodiment, the cell viability (of a Lactiplantibacillus specie, such as Lactobacillus plantarum) has been determined herein by measuring lactate concentration within the activating composition, upon contacting thereof with the dry mat of the invention under appropriate conditions, as described herein.

In some embodiments, a w/w concentration of the water-soluble polymer within the fibrous material of the invention is between 0.1 and 20%, between 0.5 and 20%, between 0.5 and 15%, between 0.5 and 10%, between 1 and 20%, between land 15%, between 1 and 10%, between 1 and 5%, between 5 and 20% m between 5 and 15%, including any range between.

In some embodiments, a w/w concentration of the water-insoluble polymer within the fibrous material of the invention is between 2 and 30%, between 5 and 30%, between 10 and 30%, between 10 and 20%, between 2 and 20%, between 5 and 15%, between 5 and 10%, between 5 and 20%, including any range between. In some embodiments, a w/w concentration of the water-soluble polymer within the fibrous material of the invention is between about 0.5 and about 10%, and a w/w concentration of the water-insoluble polymer within the fibrous material of the invention is between 5 and 20%, including any range between.

In some embodiments, the fibrous material of the invention is an shapeable fibrous material (as described hereinbelow), wherein the shapeable fibrous material is composed essentially of the polymeric fibers of the invention (i.e. electrospun polymeric fibers disclosed herein), and is characterized by (i) a dry weight per weight (w/w) concentration of the encapsulated cells in a range between about 10 and about 95%, between about 10 and about 93%, between about 10 and about 90%, between about 10 and about 80%, including any range between; (ii) a thickness between 10 um and about 2000 um, or between 10 and about 1500 um; and by (iii) average fiber diameter between about 10 and about 300 um, or between about 10 and about 200 um.

In some embodiments, at least 85%, at least 90%, at least 95%, at least 99%, or between 85 and 95%, between 85 and 100%, between 90 and 10%, between 95 and 99% from the dry weight of the shapeable fibrous material of the invention consists of the polymeric fibers of the invention. In some embodiments, the shapeable fibrous material of the invention is further characterized by a water absorption capability of up to 1000%, or up to about 700%, relative to the dry weight of the material and by a tensile strength of at least 1 MPa, at least 2 MPa, at least 5 MPa, at least 7 MPa, at least 10 MPa, between 2 and 100 MPa, between 2 and 50 MPa, between 2 and 10 MPa, between 2 and 20 MPa, including any range between.

In some embodiments, the fibrous material further comprises an edible matter. In some embodiments, the mat further comprises a plant material. In some embodiments, the mat further comprises a plant extract. In some embodiments, the plant material is or comprises a cosmeceutical active ingredient. In some embodiments, the mat further comprises a metabolite (such as a cell metabolite, a plant metabolite, a bacterial metabolite, a fungal metabolite or any combination thereof). In some embodiments, the fibrous material is composed essentially of biocompatible materials or cosmeceutical grade materials. In some embodiments, the core of the polymeric fibers mat is composed essentially of biocompatible materials or cosmeceutical grade materials. In some embodiments, the core of the polymeric fibers mat is composed essentially of natural-based or naturally derived compounds or constituents.

In some embodiments, the fibrous material of the invention is characterized by a pleasant touch feeling, when applied on the skin of the subject.

In some embodiments, the fibrous material of the invention is characterized by a water absorption capability of between 50 and 5000%, between 50 and about 1000%, between 50 and 800%, between 50 and 700%, between about 50 and 100%, between 100 and 5000%, between 100 and 500%, between 500 and 1000%, between 1000 and 5000%, between 1000 and 3000%, 800%, up to 700%, up to 600%, between 3000 and 5000%, including any range between. In some embodiments, the fibrous material of the invention is characterized by a water absorption capability of between 50 and about 1000%, between 50 and 800%, between 50 and 700%, between about 50 and 500%, including any range between. In some embodiments, the water absorption is relative to the dry weight of the mat.

In some embodiments, the fibrous material is in a form of a cosmeceutical product, of a cosmetic product, or a cosmetic article. In some embodiments, the fibrous material in a form of a cosmetic/cosmeceutical product or a cosmetic/cosmeceutical article is an elastic fibrous material. In some embodiments, the fibrous material in a form of a cosmetic/cosmeceutical product or a cosmetic/cosmeceutical article is a shapeable fibrous material.

In some embodiments, the shapeable fibrous material encompasses a flexible, elastic, and/or deformable material capable for obtaining a predetermined shape. In some embodiments, the shapeable fibrous material in a moist state is configured to obtain a predetermined shape and to retain its shape for a predetermined time period (e.g. between 1 minute and 10 hours). In some embodiments, shapeable fibrous material in a moist state is characterized by capillary adhesion to the application site. In some embodiments, the predetermined shape is substantially the shape of the application site (e.g. a region within the skin of the subject, such as face, palm, feet, etc.,). In some embodiments, the shapeable fibrous material in a moist state is configured for binding to that application site by substantially obtaining the shape of the application site, wherein binding is for predetermined time period, as described above.

In some embodiments, the shapeable fibrous material is characterized by elasticity sufficient for application thereof at the target site. In some embodiments, the shapeable fibrous material is characterized by elasticity sufficient for substantially obtaining the shape of the application site, wherein substantially is at least 80%, at least 90%, at least 95% shape similarity, including any range between. In some embodiments, the cosmetic product is a ski mask (e.g. a face mask) comprising or consisting essentially of the shapeable fibrous material as disclosed hereinabove. In some embodiments, the cosmetic product has a width and a length dimension compatible with the dimensions of the application site. In some embodiments, the cosmetic product has a width and a length dimension sufficient for at least 70%, at least 80%, at least 85%, at least 90%, at least 95%, at least 99% coverage of the target site. In some embodiments, the target site refers to the application site on top of the skin of a subject. In some embodiments, the target site comprises any site on top of the human body. In some embodiments, the cosmetic product has a width and a length dimension compatible with the dimensions of the application site and further has a thickness between 10 and about 2000 um.

In some embodiments, the cosmetic product is in a form of a uniform layer, or a sheet. In some embodiments, the cosmetic product has at least one dimension (length and/or width) ranging between 1 and 50 cm, between 1 and 10 cm, between 5 and 10 cm, between 1 and 30 cm, between 1 and 20 cm, between 10 and 50 cm, between 10 and 20 cm, between 20 and 50 cm, between 30 and 50 cm, including any range between.

In some embodiments, the cosmetic product comprises a moist mat of the invention. In some embodiments, the cosmetic product comprises the mat of the invention soaked with or wetted by the activation composition. In some embodiments, the moist mat has a sufficient moisture for (i) activating the dormant cells. In some embodiments, the moist mat has a sufficient moisture for (ii) supporting free diffusion of a cosmeceutical active agent from the core towards the outer portion of the cosmeceutical product, wherein the outer portion is in contact with the skin (target site). In some embodiments, the moist mat has a sufficient moisture for supplementing the skin with a cosmeceutically effective amount of the cosmeceutical active agent. In some embodiments, the moist mat has a sufficient moisture for inducing a capillary force sufficient for a stable attachment of the moist mat to the skin of the subject (target site).

In some embodiments, the cosmetic product comprising or consisting essentially of a moist mat comprising cells in the active state encapsulated therewithin, refers to herein as a ready to use cosmeceutical product. In some embodiments, the dormant cells are activated upon contacting the dry mat with the activating composition under appropriate conditions, as described herein. In some embodiments, the ready to use cosmetic product comprises at least 30%, at least 40%, at least 50%, at least 60%, at least 70%, at least 80%, at least 90%, at least 95%, at least 97%, at least 99% of activated cells, wherein the percentage of activated cells is relative to the amount of the dormant cells within dray mat of the invention.

In some embodiments, the cosmetic product is configured for application to the target site. In some embodiments, the cosmetic product has dimensions sufficient for a substantial coverage of the target site. In some embodiments, the cosmetic product is characterized by a flexibility or elasticity sufficient to substantially adopt the 3D shape and/or configuration of the target site. In some embodiments, the cosmetic product is characterized by a flexibility or elasticity sufficient for contacting the target site or for substantial attachment to the target site.

In some embodiments, the cosmetic product (e.g. a moist mat) is characterized by adhesiveness to the target site. In some embodiments, the cosmetic product (e.g. a moist mat) is characterized by adhesiveness sufficient for stably attaching or adhering to the skin (e.g. target site). In some embodiments, the adhesiveness of the cosmetic product (e.g. a moist mat) is sufficient to retain the mat substantially in contact with the skin (or bound/attached to the skin) at the target site when (i) the skin is subjected to deformation (e.g. stretching, folding, etc.) and/or (ii) upon movement of at least a part of the subject's body. In some embodiments, the adhesiveness of the cosmetic product (e.g. a moist mat) is sufficient to withstand gravity forces and/or shear forces (e.g. due to the deformation of the subject's body or of the subject's skin, and/or due to the friction between the parts of the subject's body). In some embodiments, adhesiveness is induced by capillary forces between the wet mat and the skin in contact therewith.

In some embodiments, the adhesiveness of the cosmetic product (e.g. a moist mat) is sufficient to retain the mat substantially attached (or adhered, or bound) to the target site for a time period of between 10 minutes (min) and 24 hours (h), between 10 min and 1 h, between 1 h and 24 h, between 1 h and 2 h, between 2 h and 5 h, between 5 h and 10 h, between 10 h and 24 h, including any range between.

In some embodiments, the moisture content of the cosmetic product (e.g. a moist mat) is greater by at least 10%, at least 30%, at least 50%, at least 100%, at least 150%, at least 200%, at least 500%, by at least 1000% than a moisture content of the dry mat of the invention.

In some embodiments, the moisture content of the cosmetic product (e.g. a moist mat) is between 5 and 5000%, between 5 and 10%, between 10 and 300%, between 15 and 300%, between 20 and 300%, between 20 and 30%, between 25 and 300%, between 30 and 50%, between 50 and 100%, between 100 and 150%, between 150 and 200%, between 250 and 300% w/w, between 300 and 500% w/w, between 500 and 1000% w/w, between 1000 and 2000% w/w, between 2000 and 3000% w/w, between 3000 and 5000% w/w, including any range between. In some embodiments, the moisture content of the moist substrate of the invention is up to 800%, up to 700%, up to 600% including any range between.

In some embodiments, the mat of the invention has a sufficient mechanical strength to remain substantially intact upon wetting thereof (e.g., so as to obtain the moist mat characterized by a moisture content as disclosed herein). In some embodiments, the substantially intact mat refers to a mat which retains about 80%, about 90%, about 95%, about 99% or more of at least one dimension thereof.). In some embodiments, the substantially intact mat refers to a mat which retains about 80%, about 90%, about 95%, about 99% or more of its functional properties (e.g. surface coverage, mechanical intactness, two dimensional shape, adhesiveness, encapsulated cell content, cell viability or any combination thereof). In some embodiments, sufficient mechanical strength refers to a tensile strength of at least 1 MPa, at least 2 MPa, at least 5 MPa, at least 7 MPa, at least 10 MPa, between 2 and 100 MPa, between 2 and 50 MPa, between 2 and 10 MPa, between 2 and 20 MPa, including any range between. In some embodiments, the cosmetic product (e.g., a moist mat) is further adopted for subsequent removal thereof form the target site. In some embodiments, the moist mat remains substantially intact upon removal or detachment thereof from the target site.

In some embodiments, the substantially intact mat of the invention refers to a mat which substantially retains its mechanical strength (e.g. doesn't tear or doesn't disassemble) and/or dimensions upon wetting thereof and/or applying thereof to a skin and/or removing thereof from the skin of the subject (e.g. to the target site). In some embodiments, the substantially intact mat of the invention refers to a moist mat which doesn't tear upon subjecting thereof to a deformation stress equivalent to the stress emerging by applying and/or removing the moist mat from the target site.

In some embodiments, the activating composition is a liquid composition. In some embodiments, the activating composition is an aqueous composition. In some embodiments, the activating composition is a plant-derived liquid composition. In some embodiments, the activating composition comprises a plant matter. In some embodiments, the activating composition comprises a plant extract. In some embodiments, the activating composition comprises one or more nutrients in an amount sufficient for activating the dormant cells. In some embodiments, the activating composition is capable of activating the dormant cells. In some embodiments, the activating composition is capable of activating the cells encapsulated within the mat of the invention.

In some embodiments, the activating composition comprises a liquid (e.g., a juice or an extract) derived from a plant or a plant part. In some embodiments, the activating composition comprises a cell extract (e.g. a yest extract). In some embodiments, the activating composition consists essentially of natural compounds or natural constituents. In some embodiments, the activating composition consists essentially of plant-based compounds or plant-based constituents. In some embodiments, the activating composition comprises a growth medium (such as MRS, which is known in the art).

In some embodiments, the activating composition is capable of enhancing cell activation by at least 5%, at least 10%, at least 15%, at least 30%, at least 40%, at least 50%, at least 100%, at least 200%, as compared to a control including any range between. In some embodiments, the cell activation increase is calculated based on the cell activity assessed upon activating of the dormant cells with an activating composition, wherein the cell activity is determined as described hereinabove. In some embodiments, the control is MRS medium.

Non-limiting examples of the plant-based constituents and plant extracts include but are not limited to: moringa extract, maca extract, chlorella extract, soy extract, apple syrup, apple juice, whole apple, nettle extract, wheatgrass extract, whole grapes, grape juice, grape extract, coconut liquid, pomegranate juice, agave syrup, agave extract, aloe vera syrup or extract, beet root juice or extract, carob extract or syrup thereof, green tea extract, maple syrup, calendula extract, vitania extract, cocoa extract, whole cocoa, honey, whole cucumber, cucumber juice or extract, whole dates, date syrup, whole blueberries, blueberry juice or extract, including any fraction thereof, any extract or combination thereof.

In some embodiments, the activating composition further comprises between 0.01 and 50% w/w of any one of a nutrient, a mineral (e.g. a metal carbonate salt, a buffering agent, tri sodium phosphate, etc.), a sugar (mono-, di-, oligo-, and/or polysaccharide), molasses, a surface active compound (e.g. tween surfactant) or any combination thereof.

In some embodiments, the activating composition is characterized by pH between 3 and 10, between 3 and 5, between 5 and 7, between 5 and 10, between 7 and 8, between 8 and 10, including any range between.

### Method of Manufacturing

As used herein the phrase “co-electrospinning” refers to a process in which at least two immiscible polymeric solutions are electrospun from co-axial capillaries (i.e., at least two capillary dispensers wherein one capillary is placed within the other capillary while sharing a co-axial orientation) forming the spinneret within an electrostatic field in a direction of a collector. The capillary can be, for example, a syringe with a metal needle or a bath provided with one or more capillary apertures from which the polymeric solution can be extruded, e.g., under the action of hydrostatic pressure, mechanical pressure, air pressure and/or high voltage.

The collector serves for collecting the electrospun element (e.g., the electrospun microtube) thereupon. Such a collector can be a rotating collector or a static (non-rotating) collector. When a rotating collector is used, such a collector may have a cylindrical shape (e.g., a drum), however, the rotating collector can be also of a planar geometry (e.g., a horizontal disk). The spinneret is typically connected to a source of high voltage, such as of positive polarity, while the collector is grounded, thus forming an electrostatic field between the dispensing capillary (dispenser) and the collector. Alternatively, the spinneret can be grounded while the collector is connected to a source of high voltage, such as with negative polarity. As will be appreciated by one ordinarily skilled in the art, any of the above configurations establishes motion of a positively charged jet from the spinneret to the collector. Reverse polarity for establishing motions of a negatively charged jet from the spinneret to the collector are also contemplated.

For electrospinning, a first polymeric solution is injected into the inner capillary of the co-axial capillaries while the second polymeric solution is injected into the outer capillary of the co-axial capillaries. In some embodiments, in order to form a polymeric fiber (i.e., an electrospun microfiber, as mentioned above), the solvents of the second polymeric solution (which is for forming the shell of the polymeric fiber) and of the first polymeric solution (also referred herein as a core polymeric solution) have to be immiscible with each other. In some embodiments, the first polymeric solution and the second polymeric solution are immiscible. In some embodiments, the solvent of the second polymeric solution is incapable of dissolving the constituents of the first polymeric solution or vice versa.

In some embodiments, the first polymeric solution is an aqueous solution comprising an aqueous solvent, the water-soluble material and the plurality of cells and/or the plurality of spores. In some embodiments, the first polymeric solution comprises between 1 and 70%, between 1 and 10%, between 1 and 20%, between 10 and 50%, between 20 and 70%, between 10 and 70%, between 30 and 70%, between 10 and 50%, between 50 and 70%, between 20 and 50%, w/w of the water-soluble material.

In some embodiments, the first polymeric solution comprises between 0.01 and 30%, between 0.1 and 30%, between 0.5 and 30%, between 1 and 30%, between 1 and about 20%, between 1 and about 30% w/w of the water soluble polymer.

In some embodiments, the solvent of the second polymeric solution is water immiscible and comprises one or more solvents selected from THF, DMF, acetone, DMAC, chloroform, DMSO, DCM, NMP, TCE, TFE, butanol, methanol, ethanol, HFP, Isopropanol, Ethyl acetate, Ethanolamine, pentane, ethylene glycol, Diethyl ether, butyronitrile, acetonitrile, chlorobenzene, including any mixture thereof.

In some embodiments, the solvent of the second polymeric solution comprises a mixture of THF and DMF, wherein a ratio between THF and DMF is about 7:3 (v/v or w/w). In some embodiments, a v/v or w/w ratio between THF and DMF within the first polymeric solution is between 6:3 and 8:3, between 6:3 and 6.5:3, between 6.5:3 and 7:3, between 7:3 and 7.5:3, between 7.5:3 and 8:3, including any range between.

In some embodiments, the second polymeric solution comprises between 1 and 50%, between 1 and 10%, between 1 and 20%, between 10 and 50%, between 20 and 50%, between 10 and 50%, between 30 and 50%, between 20 and 50% w/w of the water-insoluble polymer.

In some embodiments, a v/v ratio between the first polymeric solution and the second polymeric solution suitable for implementation in the co-electrospinning process is between 1:3 and 3:1, including any range between.

The viscosity of the first and second polymeric solutions are compatible with each other, so as to obtain stable electrospun fibers. In some embodiments, the viscosity of the first polymeric solution and the viscosity of the second polymeric solution are substantially the same (e.g. having a variance of up to 30%, up to 20%, up to 10%, up to 5%, up to 1%, including any range between).

According to some embodiments of the invention, the solvent of the second polymeric solution is capable of evaporating through the internal surface of the shell. Exemplary co-electrospinning solutions (a first and a second solution) are as disclosed in the Examples section herein.

In some embodiments, the co-electrospinning is performed under suitable conditions comprising a flow rate of the first aqueous solution and/or of the second solution between 1 and 150 ml/h, between 10 and 150 ml/h, between 50 and 150 ml/h, between 1 and 50 ml/h, between 1 and 100 ml/h, between 10 and 100 ml/h, per single fiber spinning unit.

The flow rates of the first and second polymeric solutions can determine the microtube outer and inner diameter and thickness of shell. Non-limiting exemplary electrospinning conditions are as disclosed in any one of WO2008/041183, WO 2009/104174, WO 2009/104176 the contents of which are incorporated herein in their entirety.

### Kit

In another aspect of the invention, there is provided a kit comprising the dry mat disclosed herein, wherein the dry mat is packaged within a container. In some embodiments, the dry mat is the mat of the invention, such as comprising dormant/viable cells encapsulated therewithin. In some embodiments, a mat is the mat of the invention, such as comprising live-active (i.e., not dormant cells) encapsulated therewithin.

In some embodiments, the container further comprises a composition (e.g., an inert composition) such as for carrying the mat of the invention or media secreted therefrom (e.g., following activation of the cells encapsulated within the mat).

In some embodiments, the container has dimensions (e.g. width, length, height dimension) compatible with the corresponding dimensions of the dry mat. In some embodiments, the container comprises the dry mat in a folded state. In some embodiments, the container has dimensions (e.g. width, length, height dimension) compatible with the corresponding dimensions of the dry mat wherein the mat is in a folded state, in the unfolded state or in a partially expanded state. In some embodiments, the container is a sealed container. In some embodiments, the container comprises at least one wall defining a lumen, wherein the inner volume of the container has dimensions compatible with the corresponding dimensions of the dry mat. In some embodiments, the wall of the container is gas (e.g., an atmospheric gas, such as oxygen) impermeable. In some embodiments, the wall of the container is moisture impermeable. In some embodiments, the container comprises of two or more containers one within (inside) the other. In some embodiments, the container is configured to protect the mat of the invention from exposure to the ambient (e.g. oxygen and/or moisture). In some embodiments, the container is configured to substantially prolong the shelf life of the mat, compared to a pristine mat devoid of the packaging. In some embodiments, the container may include additional compartment and solutions supporting the mat activation, and/or additional solutions that be used before or after the matrix application.

In some embodiments, the wall of the container is characterized by a sufficient physical stability (e.g. mechanical strength) to remain intact upon subjecting the container to ambient conditions (also used herein as appropriate storage conditions). In some embodiments, the appropriate storage conditions comprise a temperature of less than 100° C., less than 50° C., less than 10° C., less than 5° C., a normal atmospheric pressure or vacuum, and optionally ambient atmosphere and UV or IR-exposure, for a time period of between 1 day and 5 years, between 1 month and 1 y, between 1 and 2 y, between 2 and 5 y including any range between.

In some embodiments, the wall of the container is characterized by physical stability (e.g. substantially retains its shape, dimension, mechanical properties) and/or chemical stability (e.g. chemically inert) upon exposure thereof to a temperature up to 60° C., up to 55° C., up to 50° C., up to 40° C., up to 30° C., including any range between.

In some embodiments, the wall of the container is characterized by a sufficient heat transfer capacity to allow an efficient heat transfer from the heat source in contact with an outer portion of the wall to the inner volume (or lumen) of the container enclosed by the wall. In some embodiments, the lumen of the container is filled with a liquid, and the wall of the container is characterized by a sufficient heat transfer capacity to allow an efficient heat transfer from the heat source in contact with an outer portion of the wall to the liquid. In some embodiments, a sufficient heat transfer capacity is so as to allow heating of the mat and the liquid to a predetermined temperature (e.g. about 35° C., about 40° C., about 45° C., about 50° C., about 55° C., about 60° C., including any range between) within a time period ranging between 10 and 60 min, between 10 and 30 min, between 30 and 60 min, between 30 and 50 min, between 40 and 60 min, including any range between.

In some embodiments, a sufficient heat transfer capacity is so as to allow heating of the mat and the liquid to a predetermined temperature (e.g. about 35° C., about 40° C., about 45° C., about 50° C., about 55° C., about 60° C., including any range between) within a time period ranging between 1 and 3 hours, between 4 and 6 hours, between 6 and 8 hours and more.

In some embodiments, the wall comprises a laminate. In some embodiments, the wall comprises a thermoplastic or a thermoset polymer. In some embodiments, the wall comprises a material suitable for manufacturing of a pouch (e.g. a polyolefin, a polyethylene vinyl alcohol (EVOH), polyethylene terephthalate (PET), polypropylene (PP) and optionally an aluminum foil, including any combinations thereof). Additional gas tight materials, having the above disclosed physical properties are well-known in the art.

In some embodiments, the container is under vacuum or is filled with an inert gas. In some embodiments, the kit (e.g. the dry mat of the invention packaged within the container) is characterized by a shelf-life of at least 1 m, at least 3 m, at least 6 m, at least 9 m, at least 12 m, at least 15 m, at least 24 m, including any range between. In some embodiments, the shelf life of the dry mat refers to the ability of the mat to substantially retain viability of the dormant cells, and/or to substantially retain physical stability and/or functional properties (including wettability, elasticity, skin adhesiveness, mechanical strength, etc.), wherein substantially is as compared to the initial cell viability physical stability and/or functional properties of the mat immediately after manufacturing thereof.

In some embodiments, the container (also referred to herein as “a first container”) comprises a seal. In some embodiments, the seal is configured to be in a close state or in an open state. In some embodiments, the seal is resealable. In some embodiments, the seal in the closed state is configured to seal the container. In some embodiments, the seal in the close state is gas tight and/or liquid/water vapor impermeable. In some embodiments, the seal is a breakable seal or a removable seal. In some embodiments, the seal is in a form of a lid, or a valve. In some embodiments, the seal in the open state is configured to support a liquid flow. In some embodiments, a liquid can be introduced to the lumen of the container via the seal in an open state.

In some embodiments, the kit further comprises an activating composition of the invention. In some embodiments, the activating composition is a liquid. In some embodiments the activating composition is stored within a second container. In some embodiments, the second container is configured for containing a liquid. In some embodiments, the second container is a liquid and/or gas tight container.

In some embodiments, the amount of the activating composition within the kit is sufficient for activating the cells within the mat of the invention. In some embodiments, a single kit comprises sufficient amount of the activating composition is so as to activate a single mat of the invention.

In some embodiments, sufficient amount of the activating composition (e.g., of the liquid composition) is between 0.1 and 10 ml, between 0.1 and 1 ml, between 0.1 and 0.5 ml, between 0.5 and 1 ml, between 1 and 2 ml, between 2 and 5 ml, between 5 and 7 ml, between 7 and 10 ml, between 10 and 15 ml, between 15 and 20 ml including any range between. A skilled artisan will appreciate that the exact amount of the activating composition may vary, depending on the dimensions of the dry mat and its composition wetting properties.

In some embodiments, the kit is configured for contacting the dry mat with the activating composition. In some embodiments, the kit is configured for contacting the wet mat with activity boosting composition. In some embodiments, the seal is configured to support or enable liquid flow (e.g. flow of the sufficient amount of the activating composition, wherein sufficient amount is as described herein). In some embodiments, the seal is configured to facilitate introduction of the activating composition into the first container comprising the dry mat. In some embodiments, the kit is configured for introducing the activating composition from the second container into the first container comprising the dry mat. In some embodiments, the liquid activating composition from the second container is introduced into the first container via a removable or a breakable seal.

In some embodiments, the first container and the second container are bound together within the kit. In some embodiments, the kit is configured for substantially providing the liquid activating composition from the second container into the first container, thereby filling the lumen of the first container with the activating composition sufficient for wetting the dry mat and for activating the dormant cells. In some embodiments, the seal is resealable so as to enable a liquid tight sealing of the first container comprising the mat in contact with the liquid activating composition.

In some embodiments, the kit further comprises a heating device. In some embodiments, the heating device is compatible with the first container of the kit. In some embodiments, the heating device is adopted for providing the moist mat within the first container under appropriate conditions. In some embodiments, the heating device comprises a heating surface compatible with the dimensions of at least one wall of the first container. In some embodiments, the dimensions of heating surface are substantially the same as the dimensions of at least one wall of the first container. In some embodiments, the heating surface is adopted for heating the first container. In some embodiments, the dimensions of heating surface are sufficient for holding the first container or for attaching the first container with the heating surface for a time period described hereinbelow.

In some embodiments, the heating device is a portable device configured for application on the target site of the subject. In some embodiments, the heating device is suitable for application on a skin of the subject. In some embodiments, the heating device is configured to generate controlled heat emission so as to induce activation of the encapsulated cells, wherein heat emission doesn't exceed a temperature harmful to a skin of the subject (e.g., doesn't exceed a temperature of 60° C., of 50° C., of 40° C., for a time period greater than 5 seconds). In some embodiments, the heating device has dimensions compatible with the dimensions of the mat and/or with the dimensions of the target site. In some embodiments, the heating device has sufficient flexibility so as to substantially obtain the shape of the target site, wherein substantially refers to at least 80%, at least 90%, at least 95% shape similarity to the target site, including any range between. In an exemplary embodiment, the heating device of the invention is configured for application on top of the moist mat at the target site, so as to induce sufficient activation of the encapsulated cells in-situ.

In some embodiments, the kit is configured for exposing the mat in contact with the liquid activating composition under condition sufficient for substantial activation of the dormant cells (also referred to herein as “appropriate conditions”). In some embodiments, appropriate conditions comprise providing the liquid activating composition to a predetermined temperature of about 35° C., about 40° C., about 45° C., about 50° C., about 55° C., including any range between. In some embodiments, appropriate conditions comprise a temperature between 3° and 55° C., including any range between. In some embodiments, appropriate conditions comprise a time period (or heating time) ranging between 10 and 180 min, between 10 and 30 min, between 30 and 60 min, between 30 and 50 min, between 40 and 60 min, between 60 and 100 min, between 100 and 120 min, between 120 and 150 min, between 150 and 180 min, between 60 and 120 min, between 60 and 180 min, including any range between.

In some embodiments, the kit is configured for facilitating heating the sealed first container comprising the mat in contact with the liquid activating composition.

In some embodiments, the kit is configured to facilitate activation of the cells within the mat, so as to obtain a ready to use cosmeceutical product. In some embodiments, the kit is configured to support activation of the dormant cells within the dry mat, by (i) providing the liquid activating composition from the second container into the first container, thereby obtaining a moist mat of the invention in contact with the activating composition; and (ii) by providing the moist mat under appropriate conditions sufficient for substantial activation of the dormant cells.

### Methods

In another aspect, there is provided a method for obtaining the ready to use cosmetic product of the invention, the method comprising contacting the dry mat disclosed herein with a sufficient amount of the activating composition under suitable conditions. In some embodiments, the suitable conditions are sufficient for activating the dormant cells, as disclosed herein. In some embodiments, the dry mat is the dry mat of the invention. In some embodiments, the dry mat is a part of the kit of the invention (e.g. packaged or stored within the first container). In some embodiments, the activating composition is the activating composition of the invention. In some embodiments, the activating composition is packaged or stored in the second container.

In some embodiments, the method comprising contacting the activating composition of the kit disclosed herein with the dry mat packaged or stored within the first container of the kit, thereby obtaining the ready to use cosmetic product of the invention. In some embodiments, contacting comprises breaking/removing the seal (or opening the valve) of the first container, and introducing the liquid activating composition into the first container via the seal or via the valve in an open state. In some embodiments, the method further comprises sealing the first container (e.g. by providing the sela or valve in the close state), to obtained a sealed container. In some embodiments, the method further comprises exposing the sealed container under conditions suitable for substantial activation of the dormant cells (e.g. a temperature between about 30 and about 55° C., and heating time between 10 min and 24 h, as disclosed herein). In some embodiments, the method further comprises removing the wet mat comprising activated cells from the first container, thereby obtaining the ready to use cosmetic product of the invention.

In another aspect, there is provided a method for in-situ generation of a cell metabolite, the method comprises contacting the dry fibrous material of the invention with an activating composition under appropriate conditions sufficient for activating the dormant cells, thereby inducing in-situ generation of the cell metabolite; wherein the activating composition is a liquid comprising capable of providing the dormant cells from the dormant state into an active state.

In some embodiments, the method for in-situ generation of a cell metabolite comprises contacting the dry mat of the kit with the activating composition of the kit, as disclosed hereinabove.

In some embodiments, there is a method for supplementing a skin of the subject with a metabolite (e.g. a cosmeceutically active metabolite), the method comprising contacting the dry mat of the kit of the invention with the activating composition of the kit of the invention under appropriate conditions, thereby obtaining a cosmetic product comprising a metabolite; and applying the cosmetic product at a target site on the skin of a subject, thereby supplementing the skin with the metabolite. In some embodiments, contacting is as described hereinabove. In some embodiments, the cosmetic product comprising a metabolite is the ready to use cosmetic product of the invention. In some embodiments, the cosmetic product comprising a metabolite is a wet mat, or a solution of the activating composition comprising the metabolite.

In some embodiments, applying the cosmetic product comprising attaching the cosmetic product to a target site on the skin of a subject. In some embodiments, the cosmetic product is remained at the target site for a sufficient time period ranging between 1 min and 2 h, including any rang between. In some embodiments, the method further comprising subsequently removing the cosmetic product from the target site.

### Definitions

As used herein, the term “consisting essentially of” means that the composition, method or structure may include additional ingredients, steps and/or parts, but only if the additional ingredients, steps and/or parts do not materially alter the basic and novel characteristics of the claimed composition, method or structure. Further, the term “consisting essentially of” means that the composition, the article (e.g., cosmetic article), the fiber, or the material of the invention is substantially composed of the constituents disclosed herein.

As used herein, the term “substantially” refers to at least 85%, at least 90%, at least 92%, at least 95%, at least 96%, at least 97%, at least 98%, at least 99%, or between 60 and 99.9%, between 70 and 80%, between 70 and 90%, between 80 and 90%, between 90 and 95%, between 95 and 99.9%, including any range or value therebetween.

As used herein, the term “substituent” encompasses hydrogen, halogen, —NO₂, —CN, —OH, oxo, imino, —CONH₂, —CONR′₂, —CNNR′₂, —CSNR′₂, —CONH—OH, —CONH—NH₂, —NHCOR, —NHCSR, —NHCNR, —NC(═O)OR, —NC(═O)NR′, —NC(═S)OR′, —NC(═S)NR′, —SO₂R′, —SOR′, —SR′, —SO₂OR′, —SO₂N(R′)₂, —NHNR′₂, —NNR′, C₁-C₆ haloalkyl, optionally substituted C₁-C₆ alkyl, —NH₂, —NR′₂, —NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, —CONH(C₁-C₆ alkyl), —CON(C₁-C₆ alkyl)₂, —CO₂H, —CO₂R′, —OCOR, —OCOR′, —OC(═O)OR′, —OC(═O)NR′, —OC(═S)OR′, —OC(═S)NR′, or a combination thereof; wherein each R′ independently represents hydrogen, or is selected from the group comprising optionally substituted C₁-C₁₀ alkyl, optionally substituted C₃-C₁₀ cycloalkyl, optionally substituted C₃-C₁₀ heterocyclyl, optionally substituted heteroaryl, optionally substituted aryl, hydroxy, amino, —NH₂, —NR′₂—NH(C₁-C₆ alkyl), —N(C₁-C₆ alkyl)₂, C₁-C₆ alkoxy, C₁-C₆ haloalkoxy, hydroxy(C₁-C₆ alkyl), hydroxy(C₁-C₆ alkoxy), alkoxy(C₁-C₆ alkyl), alkoxy(C₁-C₆ alkoxy), C₁-C₆ alkyl-NR′₂, C₁-C₆ alkyl-SR′, or a combination thereof.

As used herein, the term “alkyl” describes an aliphatic hydrocarbon including straight chain and branched chain groups. The term “alkyl”, as used herein, also encompasses saturated or unsaturated hydrocarbon, hence this term further encompasses alkenyl and alkynyl.

The term “alkenyl” describes an unsaturated alkyl, as defined herein, having at least two carbon atoms and at least one carbon-carbon double bond. The alkenyl may be substituted or unsubstituted by one or more substituents, as described hereinabove.

The term “alkynyl”, as defined herein, is an unsaturated alkyl having at least two carbon atoms and at least one carbon-carbon triple bond. The alkynyl may be substituted or unsubstituted by one or more substituents, as described hereinabove.

The term “cycloalkyl” describes an all-carbon monocyclic or fused ring (i.e., rings which share an adjacent pair of carbon atoms) group where one or more of the rings does not have a completely conjugated pi-electron system. The cycloalkyl group may be substituted or unsubstituted, as indicated herein.

The term “aryl” describes an all-carbon monocyclic or fused-ring polycyclic (i.e., rings which share adjacent pairs of carbon atoms) groups having a completely conjugated pi-electron system. The aryl group may be substituted or unsubstituted, as indicated herein.

The term “alkoxy” describes both an O-alkyl and an —O-cycloalkyl group, as defined herein. The term “aryloxy” describes an —O-aryl, as defined herein.

Each of the alkyl, cycloalkyl and aryl groups in the general formulas herein may be substituted by one or more substituents, whereby each substituent group can independently be, for example, halide, alkyl, alkoxy, cycloalkyl, nitro, amino, hydroxyl, thiol, thioalkoxy, carboxy, amide, aryl and aryloxy, depending on the substituted group and its position in the molecule. Additional substituents are also contemplated.

The term “halide”, “halogen” or “halo” describes fluorine, chlorine, bromine or iodine. The term “haloalkyl” describes an alkyl group as defined herein, further substituted by one or more halide(s). The term “haloalkoxy” describes an alkoxy group as defined herein, further substituted by one or more halide(s). The term “hydroxyl” or “hydroxy” describes a —OH group. The term “mercapto” or “thiol” describes a —SH group. The term “thioalkoxy” describes both an —S-alkyl group, and a —S-cycloalkyl group, as defined herein. The term “thioaryloxy” describes both an —S-aryl and a —S-heteroaryl group, as defined herein. The term “amino” describes a —NR′R″ group, or a salt thereof, with R′ and R″ as described herein.

The term “heterocyclyl” describes a monocyclic or fused ring group having in the ring(s) one or more atoms such as nitrogen, oxygen and sulfur. The rings may also have one or more double bonds. However, the rings do not have a completely conjugated pi-electron system. Representative examples are piperidine, piperazine, tetrahydrofuran, tetrahydropyran, morpholino and the like.

The term “carboxy” describes a —C(O)OR′ group, or a carboxylate salt thereof, where R′ is hydrogen, alkyl, cycloalkyl, alkenyl, aryl, heteroaryl (bonded through a ring carbon) or heterocyclyl (bonded through a ring carbon) as defined herein.

The term “carbonyl” describes a —C(O)R′ group, where R′ is as defined hereinabove. The above-terms also encompass thio-derivatives thereof (thiocarboxy and thiocarbonyl).

The term “thiocarbonyl” describes a —C(S)R′ group, where R′ is as defined hereinabove. A “thiocarboxy” group describes a —C(S)OR′ group, where R′ is as defined herein. A “sulfinyl” group describes an —S(O)R′ group, where R′ is as defined herein. A “sulfonyl” or “sulfonate” group describes an —S(O)2R′ group, where R′ is as defined herein.

A “carbamyl” or “carbamate” group describes an —OC(O)NR′R″ group, where R′ is as defined herein and R″ is as defined for R′. A “nitro” group refers to a —NO2 group. The term “amide” as used herein encompasses C-amide and N-amide. The term “C-amide” describes a —C(O)NR′R″ end group or a —C(O)NR′-linking group, as these phrases are defined hereinabove, where R′ and R″ are as defined herein. The term “N-amide” describes a —NR″C(O)R′ end group or a —NR′C(O)— linking group, as these phrases are defined hereinabove, where R′ and R″ are as defined herein.

A “cyano” or “nitrile” group refers to a —CN group. The term “azo” or “diazo” describes an —N═NR′ end group or an —N═N— linking group, as these phrases are defined hereinabove, with R′ as defined hereinabove. The term “guanidine” describes a —R′NC(N)NR″R′″ end group or a —R′NC(N) NR″— linking group, as these phrases are defined hereinabove, where R′, R″ and R′ are as defined herein. As used herein, the term “azide” refers to a —N3 group. The term “sulfonamide” refers to a —S(O)2NR′R″ group, with R′ and R″ as defined herein.

The term “phosphonyl” or “phosphonate” describes an —OP(O)—(OR′)2 group, with R′ as defined hereinabove. The term “phosphinyl” describes a —PR′R″ group, with R′ and R″ as defined hereinabove. The term “alkylaryl” describes an alkyl, as defined herein, which substituted by an aryl, as described herein. An exemplary alkylaryl is benzyl.

The term “heteroaryl” describes a monocyclic or fused ring (i.e., rings which share an adjacent pair of atoms) group having in the ring(s) one or more atoms, such as, for example, nitrogen, oxygen and sulfur and, in addition, having a completely conjugated pi-electron system. As used herein, the term “heteroaryl” refers to an aromatic ring in which at least one atom forming the aromatic ring is a heteroatom. Heteroaryl rings can be foamed by three, four, five, six, seven, eight, nine and more than nine atoms. Heteroaryl groups can be optionally substituted. Examples of heteroaryl groups include, but are not limited to, aromatic C₃₋₈ heterocyclic groups containing one oxygen or sulfur atom, or two oxygen atoms, or two sulfur atoms or up to four nitrogen atoms, or a combination of one oxygen or sulfur atom and up to two nitrogen atoms, and their substituted as well as benzo- and pyrido-fused derivatives, for example, connected via one of the ring-forming carbon atoms. In certain embodiments, heteroaryl is selected from among oxazolyl, isoxazolyl, oxadiazolyl, thiazolyl, isothiazolyl, pyridinyl, pyridazinyl, pyrimidinal, pyrazinyl, indolyl, benzimidazolyl, quinolinyl, isoquinolinyl, quinazolinyl or quinoxalinyl.

In some embodiments, a heteroaryl group is selected from among pyrrolyl, furanyl (furyl), thiophenyl (thienyl), imidazolyl, pyrazolyl, 1,2,3-triazolyl, 1,2,4-triazolyl, 1,3-oxazolyl (oxazolyl), 1,2-oxazolyl (isoxazolyl), oxadiazolyl, 1,3-thiazolyl (thiazolyl), 1,2-thiazolyl (isothiazolyl), tetrazolyl, pyridinyl (pyridyl)pyridazinyl, pyrimidinyl, pyrazinyl, 1,2,3-triazinyl, 1,2,4-triazinyl, 1,3,5-triazinyl, 1,2,4,5-tetrazinyl, indazolyl, indolyl, benzothiophenyl, benzofuranyl, benzothiazolyl, benzimidazolyl, benzodioxolyl, acridinyl, quinolinyl, isoquinolinyl, quinazolinyl, quinoxalinyl, phthalazinyl, thienothiophenyl, 1,8-naphthyridinyl, other naphthyridinyls, pteridinyl or phenothiazinyl. Where the heteroaryl group includes more than one ring, each additional ring is the saturated form (perhydro form) or the partially unsaturated form (e.g., the dihydro form or tetrahydro form) or the maximally unsaturated (nonaromatic) form. The term heteroaryl thus includes bicyclic radicals in which the two rings are aromatic and bicyclic radicals in which only one ring is aromatic. Such examples of heteroaryl are include 3H-indolinyl, 2(1H)-quinolinonyl, 4-oxo-1,4-dihydroquinolinyl, 2H-1-oxoisoquinolyl, 1,2-dihydroquinolinyl, (2H)quinolinyl N-oxide, 3,4-dihydroquinolinyl, 1,2-dihydroisoquinolinyl, 3,4-dihydroisoquinolinyl, chromonyl. In some embodiments, heteroaryl groups are optionally substituted. In one embodiment, the one or more substituents are each independently selected from among halo, hydroxy, amino, cyano, nitro, alkylamido, acyl, C₁₋₆-alkyl, C₁₋₆-haloalkyl, C₁₋₆-hydroxyalkyl, C₁₋₆-aminoalkyl, C₁₋₆-alkylamino, alkylsulfenyl, alkylsulfinyl, alkylsulfonyl, sulfamoyl, or trifluoromethyl.

Examples of heteroaryl groups include, but are not limited to, unsubstituted and mono- or di-substituted derivatives of furan, benzofuran, thiophene, benzothiophene, pyrrole, pyridine, indole, oxazole, benzoxazole, isoxazole, benzisoxazole, thiazole, benzothiazole, isothiazole, imidazole, benzimidazole, pyrazole, indazole, tetrazole, quinoline, isoquinoline, pyridazine, pyrimidine, purine and pyrazine, furazan, 1,2,3-oxadiazole, 1,2,3-thiadiazole, 1,2,4-thiadiazole, triazole, benzotriazole, pteridine, phenoxazole, etc.

As used herein, the terms “halo” and “halide”, which are referred to herein interchangeably, describe an atom of a halogen, that is fluorine, chlorine, bromine or iodine, also referred to herein as fluoride, chloride, bromide and iodide.

As used herein, the term “substituted” or the term “substituent” are related to one or more (e.g., 2, 3, 4, 5, or 6) substituents, wherein the substituent(s) is as described herein.

### General

As used herein the terms “about” and “approximately” refers to ±10%.

The terms “comprises”, “comprising”, “includes”, “including”, “having” and their conjugates mean “including but not limited to”. The term “consisting of means” “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The term “consisting essentially of” means that the composition, method or structure may include additional ingredients, steps and/or parts, but only if the additional ingredients, steps and/or parts do not materially alter the basic and novel characteristics of the claimed composition, method or structure.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, electrochemical, and electronical arts.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.).

It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

### EXAMPLES

The inventors successfully manufactured hollow electrospun polymeric fibers enclosing high density of viable cells and/or microorganisms inside the fiber. The shell of these fiber is a porous shell comprising a plurality of openings. The porous shell enables diffusion of the nutrients required by the encapsulated microorganisms from the outside of the fiber, and at the same time the products (metabolites) produced by the cells and/or microorganisms are capable of diffusing out of the fiber. Fiber diameters can range from a few microns to hundreds of microns. In addition, the thickness of the porous shell is controllable, ranging from several tens of nanometers to about 1 micron, and the pore size of the shell can vary from few nano-meters to few microns.

Also, unwanted microorganisms can't enter to the fibers structure. The integrated cells/organisms are protected and effectively there is no limitation on mass transfer from and into the fibrous structures. Therefore, it is possible to secure a size that allows the nutrients and by-products to be freely exchanged from and into the fibers. In this way, similarly, fiber dimensions can be produced as designed according to the type of microorganism to be encapsulated, the nutrients it requires, and the size of by-products.

Current technology enables designing fibrous mats for various applications not only in the degree of freedom in the structure of the fiber, but also in the way the 3D structure of the mat should be. First of all, the structure of the thread (or polymeric fiber) can be controlled in size according to the microorganisms to be enclosed. Furthermore, the structure of the mat created by the fibers can be oriented and designed according to the application, or also, when desired, it can be built into a multi-layered or three-dimensional structure.

As for the microorganisms to be encapsulated, various types of cells and/or microorganisms such as various bacteria, yeast, algae, mammalian and plant cells (including stem cells derived from humans and plants) can be included in high concentration and live-active state. Additionally, enzymes and other biomolecules such as proteins and hormones, etc. can be included in active state in the final fabric. The live-state enables the activity of the cells and microorganism. As for the material of the fiber itself, one can select natural substances as well as synthetic polymers.

Furthermore, depending on the purpose of the final reaction, hydrophilic or hydrophobic materials, degradable/biodegradable or inert materials can be selected. Optionally, biocompatible and/or biodegradable materials such as medical grade constituents can be applied for the manufacturing of the mat.

Optionally, there are three areas of application of the mats disclosed herein to the skincare-cosmetics industry: Various good bacteria that can be effective for the overall skin health and various skin problems. It can be applied to a variety of applications, from general beauty masks to patches for dealing with specific skin problems.

In addition, it is possible to safely and efficiently extract the products produced by the cells encapsulated within the polymeric fibers. It can also be used as a material for bio-production utilizing this effect. Using this bioprocessing capability, we can build a bio production system that effectively produces various additives and nutrients for cosmetics and foods.

In most cases, “probiotics” in the available cosmetic products have been either based on (i) non-live or inert bacteria applied to the skin, (ii) lysate materials (certain non-fresh product resulting from the breaking down the cells), or (iii) processed probiotics growth media. Products that contain inert (non-live) bacteria, the destroyed cells products or used growth media, in cream or apply it to the surface of a face mask, have been the appearance of general probiotic cosmetics so far. In addition, the herein disclosed fibrous mats may also include supportive skin-nutrients and supper-foods.

Currently disclosed fibrous mats, on the other hand, provide a better environment for the skin with live-active bacteria protected inside the fibers. By enclosing dormant bacteria inside the fiber, it is possible to provide about 1,000 times the number of bacteria compared to conventional methods. Moreover, the cells (e.g. bacterial cells) are viable and upon activation thereof release “fresh and natural” active elements to the skin of the subject, while the bacteria contained in the fiber cannot go out.

To this end, upon activation the cells (e.g. bacterial cells) encapsulated within the fibrous material disclosed herein, are capable of releasing metabolites such as natural vitamins, acids and other various skin nutrients, which are in-situ synthesized by the cells, and thus (a) directly effect on the actual skin conditions to keep the skin moisturized, elastic and youthful, and increase resistance to ultraviolet rays and wrinkles, (b) also support the existing/surviving microbiome population. In this way, by supplying microorganisms from the inside of the electrospun polymeric fibers, it is possible to obtain a healthy microbiome which can be easily applied directly to the skin of the subject.

The inventors successfully manufactured various fibrous mats by co-electrospinning based on the non-limiting exemplary core/shell polymeric solutions listed in Table 1 below.

TABLE 1

| | Core (2^{nd} polymeric | Shell (1^{st} polymeric | |
|---|---|---|---|
| # | solution) | solution) | Remarks |
| 1 | 44% w/v glycerol; 100- | 15% w/v PVDF-HFP in | Fibrous mats with insufficient |
| | 200 gr/ml bacterial cells | DMF:THF 1:2 (V/V); 4% | shelf-life, cell death after 24 h |
| | | PEG6k | |
| 2 | 40-100% w/v sucrose; | 10-14% w/v PVDF-HFP; 1- | Stable spinning process, good |
| | 10-30% w/v maltose; 1- | 5% w/v PVP; 0.5-2% w/v | shell porosity. Fibrous mats |
| | 10% w/v PEG | DW in THF:DMF 7:3 (v/v) | with sufficient shelf-life. May |
| | | | form hollow-tubes |
| 3 | Bacterial cells; 80- | 10-14% w/v PVDF-HFP; | Stable spinning process, good |
| | 200% w/v sucrose; 10- | 1-5% w/v PVP; 0.5-2% | shell porosity. Fibrous mats |
| | 30% w/v maltose; | w/v DW in THF:DMF 7:3 | with sufficient cell viability |
| | about 0.1-2% w/v | (v/v) | (at least 3-6 months). High |
| | lambda-carrageenan | | cell loading, ratio of the |
| | | | bacterial cells to the dry core |
| | | | constituents up to about 1:1 |
| 4 | Bacterial cells; 80- | 1-30% w/v cellulose or a | Stable spinning process, good |
| | 250% w/v sugar | cellulose derivative; | shell porosity. Fibrous mats |
| | (sucrose, maltose or | optionally 1-5% w/v PVP; | with sufficient cell viability |
| | both); about 0.1-10% | in THF:DMF 7:3 (v/v) | (at least 3-6 months). High |
| | w/v of a water-soluble | Other solvent mixtures | cell loading (up to about 1012 |
| | gum (e.g., lambda- | have been also | CFU/cm²). Ratio of the |
| | carrageenan) or about | successfully implemented | bacterial cells to the dry core |
| | 10-30% PEG or PPG | | constituents up to about |
| | | | 95% w/w |

Composition 2 comprises a high MW PEG (e.g., Mw of about 400 KDa). Composition 3 comprises about 700×10⁶ CFU/ml within the 2nd polymeric solution, composition 4 comprises about 10⁶-10⁸ CFU/ml within the 2nd polymeric solution. Compositions containing less than 50% w/v sugar (e.g. sucrose and maltose) within the 2nd polymeric solution resulting in inferior mats.

Additional stable fibrous mats have been successfully manufactured by implementing between 10 and 20% w/v of a water-soluble polymer (such as PEG or hydroxyethyl cellulose, abbreviated as HEC) within the 1^{st} polymeric solution instead of PVP (composition 2).

Furthermore, the inventors successfully manufactured fibrous mats with cellulose-based shell (composition 4) by replacing PVDF-HFP of composition 3 with cellulose or a water-insoluble cellulose derivate (e.g. cellulose acetate, or ethyl cellulose).

The abovementioned cellulose-based materials (obtained from compositions 3 and 4) have been utilized to obtain shapeable/elastic electrospun fibrous mats characterized by sufficient mechanical strength and elasticity for skin application and subsequent removal, and having a microbial cell loading of up to about 10¹² CFU/cm² corresponding to w/w concentration of microbial cells in the fibrous mats of up to 90%.

Furthermore, the inventors have successfully implemented numerous water-soluble polymers in the core solution so as to obtain electrospun fibrous mats of the invention with a water-soluble polymer w/w content between about 0.2 and about 20% relative to the dry matter. Following water-soluble polymers have been implemented for the electrospinning of the fibrous mats (at a w/w concentration within the core electrospinning solution ranging between about 0.01 and 30%), and the physico-mechanical properties of the resulting mats have been scored:

| | | Arbitrary score |
|---|---|---|
| Material in core | Polymer family | [from 0 to 10] |
| Guar gum | polysaccharide | 6 |
| Locust bean gum | polysaccharide | 7 |
| Acacia gum | polysaccharide | 5 |
| Carrageenan | polysaccharide | 10 |
| PEG | Polyether synthetic | 10 |
| PVA | Polyvinyl synthetic | 7 |
| PVP | Polyvinyl synthetic | 8 |

Further, the inventors have examined the thickness of the fibrous mat in terms of suitability thereof for skin application. The fibrous mats have been scored considering numerous properties, such as user compliance (ease of application, elasticity/shapeability), mechanical strength, skin adhesion, water absorption, and transparency. The inventors observed that no mat could be obtained at a thickness below 10 um (score 0-1).

Mats with a thickness between 10 um to 1000 um (score: 10) have been characterized by sufficient flexibility when handled, superior skin adhesion and shapeability (substantially adopts the shape of the target site on the skin), and by transparency. Further, these mats have been characterized by superior water permeability and liquid absorption. Further, these mats exhibited superior bioactivity (as determined by measuring metabolism rate). To this end, mats with a thickness between about 10 um and about 1000 um are superior for cosmetic application (e.g., as a face mask).

Mats with a thickness between 1000 um to 2000 um (score: 5) have been characterized by reduced flexibility (however still flexible and provide comfortable feeling when applied to a skin). Transparency is reduced-fabric is opaque, as well as bioactivity compared to 10 um-1000 um mats (however still very active). Further, these mats have been characterized by reduced water permeability and improved liquid absorption compared to 10 um-1000 um mats. To this end, mats with a thickness between about 1000 um and 2000 um are suitable for cosmetic application (e.g. as a face mask).

Mats with a thickness above 2000 um (score 0-1) have been characterized by low flexibility (hard to handle). These mats do not adhere well to skin, are nontransparent, and have reduced bio activity. Further, these mats have been characterized by reduced water permeability and improved liquid absorption compared to 1000 um-2000 um mats.

Exemplary mats of the invention have been characterized by water absorption of up to about 700%, relative to the initial dry weight of the mat. Furthermore, the inventors have tested exemplary mats with (i) varying cell loading; (ii) varying fiber diameters, and (iii) varying pore size/porosity of the fiber's shell. W/w cell concentration in the mat has been calculated as follows.

$$\frac{Cell fraction of core w / w}{( ploymer fraction of shell w / w ) + ( Cell fraction of core w / w )} = \frac{w}{w} of cell to fabric$$

The results are summarized below:

| w/w ratio cells | | |
|---|---|---|
| to fabric [%] | Score [0-10] | Notes |
| 50 | 10 | Production process stable. Fibrous |
| | | material with superior physical |
| | | characteristics. |
| 60 | 10 | Production process stable. Fibrous |
| | | material with superior physical |
| | | characteristics. |
| 70 | 10 | Production process stable. Fibrous |
| | | material with superior physical |
| | | characteristics. |
| 80 | 8 | Production process semi-stable. |
| | | Fibrous material with sufficient |
| | | physical characteristics. |
| 90 | 4 | Production process semi-stable. |
| | | Fabrics with reduced physical strength |
| 98 | 0 | Production process unstable. No fabric |
| | | is formed. |

| Fiber cross | CFU/cm\hat{ }2 | Fiber/mat score | |
|---|---|---|---|
| section [uM] | approximation | [0 to 10] | Notes |
| 1 | 10e8 | 6 | Mat has Low |
| | | | flexibility and |
| | | | permeability |
| 10 | 10e10 | 10 | Good flexibility |
| | | | and permeability |
| 50 | 10e11 | 10 | Good flexibility |
| | | | and permeability |
| 100 | 10e11.5 | 8 | Mat is les dens. |
| | | | Reduced tensile |
| | | | strength. |
| 300 | 10e11 | 4 | Mat with reduced |
| | | | thickness, fragile |
| 500 | 10e10 | 0 | Process unstable. |
| | | | No fabric forms |

| | Average | | |
|---|---|---|---|
| Average pore | pores per | Viability/activity | |
| diam' [nM] | uM\hat{ }2 | score [0 to 10] | notes |
| 0 | 0 | 1 | Bacteria inactive (dead) |
| | | | immediately after |
| | | | production |
| 10 | 9500 | 7 | Diffusion is not effective |
| 50 | 350 | 10 | Good |
| 100 | 70 | 10 | Good |
| 100 | 10 | 4 | Less mass transfer |
| 200 | 15 | 10 | good |
| 500 | 2 | 10 | Some bacteria escapes |
| | | | encapsulation |

Based on the above, the inventors have postulated that an exemplary cosmetical product of the invention with high loading of dormant or active cells is characterized by: (i) w/w cell loading in the fibrous material up to about 90-95%; (ii) average pore size and porosity of the shell to support bacterial viability and further activation thereof of between 10-500 nm and at least about 70%, respectively; (iii) average fiber cross-section of below 500 um, or between about 10 and about 300 um.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A fibrous mat comprising a plurality of electrospun fibers, wherein each of the electrospun fibers comprising a shell encapsulating a core, wherein:
the fibrous mat is characterized by a thickness of between 10 and 2000 um;
the core comprises (i) a water-soluble material comprising a water-soluble polymer, and (ii) a plurality of cells;
the shell is a porous shell comprising a water insoluble polymer;
and wherein a loading of the plurality of cells within the fibrous mat is up to 10¹³ CFU/cm².

2. The fibrous mat of claim 1, the plurality of cells is selected from microbial cells, microbial spores, a microorganism, or a combination thereof; optionally wherein the microbial cells or microbial spores comprise one or more species of bacteria, fungi, or both, optionally wherein the plurality of cells are dormant cells.

3. The fibrous mat of claim 1, wherein a dry weight per weight (w/w) concentration of the plurality of cells within the mat is up to about 95%.

4. (canceled)

5. The fibrous mat of claim 1, wherein any one of: (i) an average cross-section of the electrospun fibers within the fibrous mat is between 1 and 500 um; (ii) the fibrous mat is composed essentially of biocompatible materials or cosmeceutical grade materials.

6. (canceled)

7. (canceled)

8. (canceled)

9. The fibrous mat of claim 1, wherein said shell comprises at least 50% of the water insoluble polymer by dry weight, and optionally further comprises a water-soluble polymer.

10. The fibrous mat of claim 1, wherein the water insoluble polymer comprises a water insoluble polysaccharide, a fluoropolymer, or PVDF-HFP, including any combination and any copolymer thereof.

11. The fibrous mat of claim 10, wherein the water insoluble polysaccharide comprises cellulose, a water insoluble cellulose derivative, including any combination and any copolymer thereof.

12. The fibrous mat of claim 11, wherein water insoluble cellulose derivative comprises cellulose acetate, cellulose acetate phthalate, methyl cellulose, ethyl cellulose, ethyl methyl cellulose, hydroxy ethyl cellulose, hydroxy propyl cellulose, carboxy methyl cellulose, hydroxypropyl methyl cellulose, including any combination and any copolymer thereof.

13. The fibrous mat of claim 1, wherein the water-soluble polymer is selected from a water-soluble polysaccharide, a polyol, a polyvinyl alcohol, polyalkyleneoxide, PVP, a polyether, including any copolymer and any combination thereof.

14. The fibrous mat of claim 1, wherein the water-soluble material further comprises a monosaccharide, a di-saccharide, an oligosaccharide, or any combination thereof.

15. The fibrous mat of claim 1, wherein the fibrous mat is (i) shapeable, and is characterized by water absorption capability of up to 1000%, relative to the dry weight of said fibrous mat, or (ii) characterized by adhesiveness to a human skin when in a moist form.

16. (canceled)

17. The fibrous mat of claim 1, further comprising a plant extract, a bacterial metabolite, a fungal metabolite, or any combination thereof.

18. The fibrous mat of claim 1, wherein the mat is characterized by a moisture content of less than 10%, and wherein the dormant cells maintain their viability within the mat for at least 3 months.

19. The fibrous mat of claim 1, wherein an average cross-section of the electrospun fibers within the fibrous mat is between about 10 and about 200 um; wherein the w/w concentration of the plurality of cells within the mat is up to 90%, or wherein a loading of the plurality of cells within the fibrous mat is up to 10¹² CFU/cm²; wherein the shell comprises cellulose, or the water insoluble cellulose derivative; and wherein the core comprises (i) at least one of a monosaccharide, a di-saccharide, and an oligosaccharide; and (ii) the water-soluble polymer selected from polyalkyleneoxide and the water soluble polysaccharide.

20. The fibrous mat of claim 19, wherein the water-soluble polysaccharide is a water-soluble gum, and wherein the polyalkyleneoxide is PEG.

21. (canceled)

22. (canceled)

23. The fibrous mat of claim 1, being in a form of a topical product configured for application to a target site on a skin of a subject, wherein the topical product is any one of: (i) characterized by a tensile strength of at least 1 MPa; (ii) configured to substantially adopt a shape of the target site; (iii) a length dimension, a width dimension or both of the topical product is substantially compatible with the dimension of the target site; (iv) configured for stably adhere to the target site for a predetermined time period; and (v) configured for subsequent removal thereof from the target site, and wherein the topical product has sufficient mechanical strength to remain intact upon removal thereof.

24. (canceled)

25. (canceled)

26. (canceled)

27. (canceled)

28. (canceled)

29. A kit comprising the fibrous mat of claim 1 packaged within a container, wherein the fibrous mat is characterized by a water content of at most 10%, and wherein a wall of the container is water impermeable and is substantially oxygen impermeable.

30. The kit of claim 29, wherein the wall of the container is any one of (i) stable at a temperature up to 60° C. and is characterized by a sufficient heat transfer capacity; (ii) defining a lumen configured to hold a liquid volume; and wherein the container further comprises seal, and wherein the seal is a removable seal or a breakable seal characterized by an open state and by a closed state; wherein the seal is in a form of a valve, and wherein in the open state the valve is configured to be in fluid communication with the water-tight container, and to support a liquid flow from the water-tight container into the lumen wherein in the closed state the valve is configured to seal the container.

31. The kit of claim 29, further comprising any one of: (i) a water-tight container comprising an activating composition, wherein the plurality of cells within the fibrous mat are substantially in a dormant state, and wherein the activating composition is capable of providing the plurality of cells from a dormant state into an active state; and (ii) a heating device.

32. The kit of claim 31, wherein the activating composition is any one of: (i) a liquid comprising an effective amount of a plant-based compound, or a plant extract, and optionally any one of a nutrient, a mineral, a mono-saccharide, a disaccharide, oligo-saccharide, a polysaccharide, a surface-active compound, or any combination thereof, optionally wherein the effective amount is sufficient for activation of dormant cells upon contacting the activating composition with the fibrous mat under operable conditions, optionally wherein the operable conditions comprise a temperature between about 30 and about 55° C.; and (ii) characterized by pH between 3 and 10.

33.-64. (canceled)
